SEQUENCE LISTING (34 sequences)

SEQ ID NO: 1 - protein, 9 aa, Dengue virus
GMLQGRGPL

SEQ ID NO: 2 - protein, 9 aa, Dengue virus
VAFLRFLTI

SEQ ID NO: 3 - protein, 8 aa, Dengue virus
RALIFILL

SEQ ID NO: 4 - protein, 8 aa, Dengue virus
MTMRCIGI

SEQ ID NO: 5 - protein, 8 aa, Dengue virus
VSWTMKIL

SEQ ID NO: 6 - protein, 8 aa, Dengue virus
FSLGVLGM

SEQ ID NO: 7 - protein, 9 aa, Dengue virus
VAVSFVTLI

SEQ ID NO: 8 - protein, 9 aa, Dengue virus
LAVTIMAIL

SEQ ID NO: 9 - protein, 9 aa, Dengue virus
TAIANQATV

SEQ ID NO: 10 - protein, 9 aa, Dengue virus
TAIANQATV

SEQ ID NO: 11 - protein, 9 aa, Dengue virus
YSQVNPITL

SEQ ID NO: 12 - protein, 9 aa, Dengue virus
RMLINRFTM

SEQ ID NO: 13 - protein, 9 aa, Dengue virus
RLITVNPIV

SEQ ID NO: 14 - protein, 9 aa, Dengue virus
KLAEAIFKL

SEQ ID NO: 15 - protein, 114 aa, Dengue virus
MNNQRKKARNTPFNMLKRERNRVSTVQQLTKRFSLGMLQGRGPLKLFMALVAFLRFLTIPPTAGILKRWGTIKKSKAINVLRGFRKEIGRMLNILNRRRRTAGMIIMLIPTVMA

SEQ ID NO: 16 - protein, 166 aa, Dengue virus
FHLTTRNGEPHMIVSRQEKGKSLLFKTGDGVNMCTLMAMDLGELCEDTITYKCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMT

SEQ ID NO: 17 - protein, 495 aa, Dengue virus
MRCIGISNRDFVEGVSGGSWVDIVLEHGSCVTTMAKNKPTLDFELIKTEAKQSATLRKYCIEAKLTNTTTESRCPTQGEPSLNEEQDKRFVCKHSMVDRGWGNGCGLFGKGGIVTCAMFTCKKNMKGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMLETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 18 - protein, 353 aa, Dengue virus
ADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLREKQDVFCDSKLMSAAIKDNRAVHADMGYWIESALNDTWKIEKASFIEVKSCHWPKSHTLWSNEVLESEMIIPKNFAGPVSQHNYRPGYHTQTAGPWHLGKLEMDFDFCEGTTVVVTEDCGNRGPSLRTTTASGKLITEWCCRSCTLPPLRYRGEDGCWYGMEIRPLKEKEENLVNSLVTA

SEQ ID NO: 19 - protein, 218 aa, Dengue virus
GHGQIDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRKMEKYQLAVTIMAILCVPNAVILQNAWKVSCTILAVVSVSPLFLTSSQQKADWIPLALTIKGLNPTAIFLTTLSRTNKKR

SEQ ID NO: 20 - protein, 130 aa, Dengue virus
SWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSLPITAAAWYLWEVKKQR

SEQ ID NO: 21 - protein, 618 aa, Dengue virus
AGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRKLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWSSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGIKNNQILEENVEVEIWTKEGERKKLKPRWLDARIYSDPLALKEFKEFAAGRK

SEQ ID NO: 22 - protein, 150 aa, Dengue virus
SLTLSLITEMGRLPTFMTQKARDALDNLAVLHTAEAGGRAYNHALSELPETLETLLLLTLLATVTGGIFLFLMSGRGIGKMTLGMCCIITASILLWYAQIQPHWIAASIILEFFLIVLLIPEPEKQRTPQDNQLTYVVIAILTVVAATMA

SEQ ID NO: 23 - protein, 248 aa, Dengue virus
NEMGFLEKTKKDLGLGSITTQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRR

SEQ ID NO: 24 - protein, 900 aa, Dengue virus
GTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFTPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRHKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTVTEEIAVQNWLARVGRERLSRMAISGDDCVVKPLDDRFASALTALNDMGKVRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTAEDMLTVWNRVWIQENPWMEDKTPVESWEEIPYLGKREDQWCGSLIGLTSRATWAKNIQTAINQVRSLIGNEEYTDYMPSMKRFRREEEEAGVLW

SEQ ID NO: 25 - protein, 114 aa, Dengue virus; identical to SEQ ID NO: 15

SEQ ID NO: 26 - protein, 166 aa, Dengue virus; identical to SEQ ID NO: 16

SEQ ID NO: 27 - protein, 495 aa, Dengue virus; identical to SEQ ID NO: 17

SEQ ID NO: 28 - protein, 353 aa, Dengue virus; identical to SEQ ID NO: 18

SEQ ID NO: 29 - protein, 218 aa, Dengue virus; identical to SEQ ID NO: 19

SEQ ID NO: 30 - protein, 130 aa, Dengue virus; identical to SEQ ID NO: 20

SEQ ID NO: 31 - protein, 618 aa, Dengue virus; identical to SEQ ID NO: 21

SEQ ID NO: 32 - protein, 150 aa, Dengue virus; identical to SEQ ID NO: 22

SEQ ID NO: 33 - protein, 248 aa, Dengue virus; identical to SEQ ID NO: 23

SEQ ID NO: 34 - protein, 900 aa, Dengue virus; identical to SEQ ID NO: 24

(19) United States
(12) Patent Application Publication
(10) Pub. No.: US 2011/0150914 A1
(43) Pub. Date: Jun. 23, 2011

(54) COMPOSITIONS AND METHODS FOR DENGUE VIRUS (DV) TREATMENT AND VACCINATION

(75) Inventors: Sujan Shresta, San Diego, CA (US); Laurene E. Yauch, La Jolla, CA (US); Alessandro Sette, La Jolla, CA (US)

(73) Assignee: LA JOLLA INSTITUTE FOR ALLERGY AND IMMUNOLOGY, La Jolla, CA (US)

(21) Appl. No.: 12/996,612

(22) PCT Filed: Jun. 9, 2009
(86) PCT No.: PCT/US09/46740

§ 371 Date: Jan. 24, 2011

Related U.S. Application Data
(60) Provisional application No. 61/060,088, filed on Jun. 9, 2008.

(51) Int. Cl. A61K 39/12; A61K 47/00; C12N 5/00; C07K 7/06; C07K 7/08; C07K 14/005; C07H 21/04; A61P 31/12

(52) U.S. Cl. 424/186.1; 424/204.1; 424/278.1; 435/325; 530/324; 530/325; 530/326; 530/327; 530/328; 530/329; 530/330; 530/350; 536/23.72

(57) ABSTRACT

The invention relates to Dengue virus (DV) peptides and compositions thereof, and methods that employ Dengue virus (DV) peptides and compositions thereof. The invention includes among other things, methods of treating Dengue virus (DV) infection or pathology, which include, for example, administering Dengue virus (DV) peptide T cell epitope, to treat a Dengue virus (DV) infection or pathology. The invention includes among other things Dengue virus (DV) vaccination and immunization methods.

### RELATED APPLICATIONS

This application claims priority to application Ser. No. 61/060,088, filed Jun. 9, 2008, and is expressly incorporated by reference in its entirety.

### GOVERNMENT SPONSORSHIP

This work was supported in part by Grants from National Institutes of Health (AI060989, AI077099-01 and U54 AI065359). The government may have certain rights in the invention.

### INTRODUCTION

Dengue virus (DENV) is a member of the Flaviviridae family, and causes an estimated 100 million cases of DF, 250,000 cases of DHF/DSS, and 25,000 deaths worldwide per year, yet currently there is no approved vaccine or antiviral treatment available (Gubler, Clin Microbiol Rev 11:480 (1998)). DF is a self-limiting yet debilitating febrile illness, whereas DHF and DSS are life-threatening and are characterized by increased vascular permeability, thrombocytopenia, hemorrhagic manifestations, and in the case of DSS, shock (ref). DENV is transmitted by the mosquitoes Aedes aegypti and Aedes albopictus, and is now endemic in more than 100 countries, including central and south America, southeast Asia, the Caribbean, and the South Pacific (WHO). The single-stranded, positive-sense RNA genome of DENV is approximately 10.7 kb and encodes three structural (core (C), envelope (E), and membrane (M)), and seven non-structural (NS) (NS1, NS2A, NS2B, NS3, NS4A, NS4B, NS5) proteins (Chambers et al., Annu Rev Microbiol 44:649 (1990)).

Infection with one DENV serotype leads to lifelong immunity against that serotype but not the other serotypes. In fact, DHF/DSS is most often observed in individuals experiencing a secondary infection with a heterologous serotype, and it has been postulated that serotype cross-reactive memory T cells and antibodies are involved in the pathogenesis (Green et al., Curr Opin Infect Dis 19:429 (2006); Halstead, Lancet 370:1644 (2007)). Thus, studies to date have focused on the pathogenic role of T cells in secondary DENV infections. For example, human studies have found that serotype cross-reactive CD8⁺ T cells are preferentially activated during secondary infection, and that these T cells demonstrate suboptimal degranulation and enhanced cytokine production (Mongkolsapaya et al., Nat Med 9:921 (2003); Mongkolsapaya et al., J Immunol 176:3821 (2006)). In contrast, little is known about the role of T cells in protection. CD8⁺ T cells are activated and functional in DENV-infected humans (Kurane et al., J Clin Invest 88:1473 (1991); Green et al., J Infect Dis 179:755 (1999); Mathew et al., J Clin Invest 98:1684 (1996)) and mice (Chen et al., J Med Virol 73:419 (2004); Beaumier et al., J Infect Dis 197:608 (2008); Rothman et al, J Virol 70:6540 (1996)), yet whether they are required for a protective host response is unknown.

### SUMMARY

The invention is based, at least in part, on Dengue virus (DV) peptides, such as a subsequence or portion of a structural core (C), membrane (M) or envelope (E) polypeptide sequence, or a non-structural (NS) NS1, NS2A, NS2B, NS3, NS4A, NS4B or NS5 polypeptide sequence. Such peptides include or consist of a subsequence or portion of structural core (C), membrane (M) or envelope (E) polypeptide sequence, or a non-structural (NS) NS1, NS2A, NS2B, NS3, NS4A, NS4B or NS5 polypeptide sequence, wherein the subsequence or portion of the Dengue virus (DV) polypeptide sequence includes a T cell epitope, which can, for example, elicit (produce, induce, increase, enhance, stimulate or activate) an anti-DV CD8⁺ T cell response in vitro or in vivo. CD8⁺ T cell responses elicited include, for example induced, increased, enhanced, stimulate or activate expression or production of a cytokine (e.g., IFN-gamma or TNF-alpha), release of a cytotoxin (perforin or granulysin), or apoptosis of a target (e.g., DENV infected) cell.

Using a predictive algorithm spanning the entire proteome, the CD8⁺ T cell response to the DENV2 strain, S221, in wild-type C57BL/6 mice, identified 12 epitopes from 6 of the 10 DENV proteins. Immunization with four immunodominant CD8⁺ T cell epitopes before S221 infection enhanced viral clearance. Anti-DENV CD8⁺ T cell response can be enhanced by immunization with DENV-specific peptides that induce cell-mediated immunity.

In accordance with the invention, there are provided Dengue virus (DV) peptides, subsequences and portions thereof, and compositions (e.g., pharmaceutical) including a Dengue virus (DV) peptide, subsequence or portion thereof, in which the peptide, subsequence or portion of the Dengue virus (DV) polypeptide sequence includes a T cell epitope. In one embodiment, a subsequence or portion includes a structural core (C), membrane (M) or envelope (E) polypeptide sequence, or a non-structural (NS) NS1, NS2A, NS2B, NS3, NS4A, NS4B or NS5 polypeptide sequence. In another embodiment, the subsequence or portion of the Dengue virus (DV) polypeptide sequence includes a T cell epitope.

A subsequence or portion of the Dengue virus (DV) polypeptide sequence includes or consists of a subsequence or portion of Dengue virus (DV) structural Core, Membrane or Envelope polypeptide sequence. Specific non-limiting examples of Dengue virus (DV) structural protein include or consist of a sequence set forth as: GMLQGRGPL (SEQ ID NO:1); VAFLRFLTI (SEQ ID NO:2); RALIFILL (SEQ ID NO:3); MTMRCIGI (SEQ ID NO:4); VSWTMKIL (SEQ ID NO:5); or RLITVNPIV (SEQ ID NO:13), or a subsequence thereof or an amino acid substitution thereof. In particular aspects, a subsequence or portion includes a T cell epitope, and elicits an anti-DV CD8⁺ T cell response (produce, induce, increase, enhance, stimulate or activate) an anti-DV CD8⁺ T cell response in vitro or in vivo, for example induced, increased, enhanced, stimulate or activate expression or production of a cytokine (e.g., IFN-gamma, TNF-alpha, IL-2, etc.), release of a cytotoxin (perforin or granulysin), or apoptosis of a target (e.g., DENV infected) cell.

A subsequence or portion of the Dengue virus (DV) polypeptide sequence includes or consists of a subsequence or portion of Dengue virus (DV) non-structural (NS) NS1, NS2A, NS2B, NS3, NS4A, NS4B or NS5 polypeptide sequence. Specific non-limiting examples of Dengue virus (DV) non-structural (NS) protein include or consist of a sequence set forth as: FSLGVLGM (SEQ ID NO:6); VAVSFVTLI (SEQ ID NO:7); LAVTIMAIL (SEQ ID NO:8); TAIANQATV (SEQ ID NO:9); TAIANQATV (SEQ ID NO:10); YSQVNPITL (SEQ ID NO:11); RMLINRFTM (SEQ ID NO:12); or KLAEAIFKL (SEQ ID NO:14), a subsequence thereof or an amino acid substitution thereof. In particular aspects, a subsequence or portion includes a T cell epitope and elicits (produce, induce, increase, enhance, stimulate or activate) an anti-DV CD8⁺ T cell response in vitro or in vivo. CD8⁺ T cell responses elicited include, for example induced, increased, enhanced, stimulate or activate expression or production of a cytokine (e.g., IFN-gamma), release of a cytotoxin (perforin or granulysin), or apoptosis of a target (e.g., DENV infected) cell.

A subsequence or portion of a Dengue virus (DV) structural core (C), membrane (M) or envelope (E) polypeptide can be a sequence identical to or derived from any Dengue virus (DV) serotype, such as a DENV1; DENV2, DENV3 or DENV4 serotype. A subsequence or portion of a Dengue virus (DV) structural core (C), membrane (M) or envelope (E) polypeptide can be a sequence having 75% or more identity to a core (C), membrane (M) or envelope (E) polypeptide of a Dengue virus (DV) serotype, such as a DENV1, DENV2, DENV3 or DENV4 serotype.

A subsequence or portion of a Dengue virus (DV) non-structural (NS) NS1, NS2A, NS2B, NS3, NS4A, NS4B or NS5 polypeptide can be a sequence identical to or derived from a Dengue virus (DV) serotypes, such as a DENV1, DENV2, DENV3 or DENV4 serotype. A subsequence or portion of a Dengue virus (DV) non-structural (NS) NS1, NS2A, NS2B, NS3, NS4A, NS4B or NS5 polypeptide can be a sequence having 75% or more identity to an NS1, NS2A, NS2B, NS3, NS4A, NS4B or NS5 polypeptide of a Dengue virus (DV) serotype, such as a DENV1, DENV2, DENV3 or DENV4 serotype.

A non-limiting representative Core sequence from which a subsequence or portion can be based upon is a sequence set forth as: MNNQRKKARNTPFNMLKRERNRVSTVQQLTKRFSLGMLQGRGPLKLFMALVAFLRFLTIPP TAGILKRWGTIKKSKAINVLRGFRKEIGRMLNILNRRRRTAGMIIMLIPTVMA. A non-limiting representative Membrane sequence from which a subsequence or portion can be based upon is a sequence set forth as: FHLTTRNGEPHMIVSRQEKGKSLLEKTGDGVNMCTLMAMDLGELCEDTITYKCPLLRQNEP EDIDCWCNSTSTWVTYGTCTTTGEHRREKRSVALVPHVGMGLETRTETWMSSEGAWKHA QRIETWILRHPGFTIMAAILAYTIGTTHFQRALIFILLTAVAPSMT. A non-limiting representative Envelope sequence from which a subsequence or portion can be based upon is a sequence set forth as: MRCIGISNRDFVEGVSGGSWVDIVLEHGSCVTTMAKNKPTLDFELIKTEAKQSATLRKYCIE AKLTNTTTESRCPTQGEPSLNEEQDKRFVCKIISMVDRGWGNGCGLFGKGGIVTCAMFTCK KNMKGKVVQPENLEYTIVITPHSGEEHAVGNDTGKHGKEIKITPQSSITEAELTGYGTVTME CSPRTGLDFNEMVLLQMENKAWLVHRQWFLDLPLPWLPGADTQGSNWIQKETLVTFKNP HAKKQDVVVLGSQEGAMHTALTGATEIQMSSGNLLFTGHLKCRLRMDKLQLKGMSYSMC TGKFKVVKEIAETQHGTIVIRVQYEGDGSPCKIPFEIMDLEKRHVLGRLITVNPIVTEKDSPV NIEAEPPFGDSYIIIGVEPGQLKLNWFKKGSSIGQMLETTMRGAKRMAILGDTAWINGSLGG VFTSIGKALHQVITGAIYOAAFSGVSWTMKILIGVIITWIGMNSRSTSLSVSLVLVGVVTLYLG VMVQA. A non-limiting representative Envelope sequence from which a subsequence or portion can be based upon is an Envelope sequence with a substitution of E₁₂₄ N→D; or E₁₂₈ K→E.

A non-limiting representative non-structural NS1 sequence from which a subsequence or portion can be based upon is a sequence set forth as: ADSGCVVSWKNKELKCGSGIFITDNVHTWTEQYKFQPESPSKLASAIQKAHEEGICGIRSVT RLENLMWKQITPELNHILSENEVKLTIMTGDIKGIMQAGKRSLRPQPTELKYSWKTWGKAK MLSTESHNQTFLIDGPETAECPNTNRAWNSLEVEDYGFGVFTTNIWLKLREKQDVFCDSKL MSAAIKDNRAVHADMGYWIESALNDTWKIEKASFIEVKSCFIWPKSHTLWSNEVLESEMIIP KNFAGPVSQHNYRPGYHTQTAGPWHLGKLEMDFDFCEGTTVVVTEDCGNRGPSLRTTTAS GKLITEWCCRSCTLPPLRYRGEDGCWYGMEIRPLKEKEENLVNSLVTA. A non-limiting representative non-structural NS2A sequence from which a subsequence or portion can be based upon is a sequence set forth as: GHGQIDNFSLGVLGMALFLEEMLRTRVGTKHAILLVAVSFVTLITGNMSFRDLGRVMVMV GATMTDDIGMGVTYLALLAAFKVRPTFAAGLLLRKLTSKELMMTTIGIVLLSQSTIPETILEL TDALALGMMVLKMVRKMEKYQLAVTIMAILCVPNAVILQNAWKVSCTILAVVSVSPLFLT SSQQKADWIPLALTIKGLNPTAIFLTTLSRTNKKR. A non-limiting representative non-structural NS2B sequence from which a subsequence or portion can be based upon is a sequence set forth as: SWPLNEAIMAVGMVSILASSLLKNDIPMTGPLVAGGLLTVCYVLTGRSADLELERAADVK WEDQAEISGSSPILSITISEDGSMSIKNEEEEQTLTILIRTGLLVISGLFPVSLPITAAAWYLWEV KKQR. A non-limiting representative non-structural NS3 sequence from which a subsequence or portion can be based upon is a sequence set forth as: AGVLWDVPSPPPVGKAELEDGAYRIKQKGILGYSQIGAGVYKEGTFHTMWHVTRGAVLM HKGKRIEPSWADVKKDLISYGGGWKLEGEWKEGEEVQVLALEPGKNPRAVQTKPGLFKTN AGTIGAVSLDFSPGTSGSPIIDKKGKVVGLYGNGVVTRSGAYVSAIAQTEKSIEDNPEIEDDIF RKRKLTIMDLHPGAGKTKRYLPAIVREAIKRGLRTLILAPTRVVAAEMEEALRGLPIRYQTP AIRAEHTGREIVDLMCHATFTMRLLSPVRVPNYNLIIMDEAHFTDPASIAARGYISTRVEMG EAAGIFMTATPPGSRDPFPQSNAPIMDEEREIPERSWSSGHEWVTDFKGKTVWFVPSIKAGN DIAACLRKNGKKVIQLSRKTFDSEYVKTRTNDWDFVVTTDISEMGANFKAERVIDPRRCMK PVILTDGEERVILAGPMPVTHSSAAQRRGRIGRNPKNENDQYIYMGEPLENDEDCAHWKEA KMLLDNINTPEGIIPSMFEPEREKVDAIDGEYRLRGEARKTFVDLMRRGDLPVWLAYRVAA EGINYADRRWCFDGIKNNQILEENVEVEIWTKEGERKKLKPRWLDARIYSDPLALKEFKEFA AGRK. A non-limiting representative non-structural NS4A sequence from which a subsequence or portion can be based upon is a sequence set forth as: SLTLSLITEMGRLPTFMTQKARDALDNLAVLHTAEAGGRAYNHALSELPETLETLLLLTLLA TVTGGIFLFLMSGRGIGKMTLGMCCITTASILLWYAQIQPHWIAASIILEFFLIVLLIPEPEKQR TPQDNQLTYVVIAILTVVAATMA. A non-limiting representative non-structural NS4B sequence from which a subsequence or portion can be based upon is a sequence set forth as: NEMGFLEKTKKDLGLGSITTQQPESNILDIDLRPASAWTLYAVATTFVTPMLRHSIENSSVN VSL [symbol] LMGLGKGWPLSKMDIGVPLLAIGCYSQVNPITLTAALFLLVAHYAIIGPG LQAKATREAQKRAAAGIMKNPTVDGITVIDLDPIPYDPKFEKQLGQVMLLVLCVTQVLMM RTTWALCEALTLATGPISTLWEGNPGRFWNTTIAVSMANIFRGSYLAGAGLLFSIMKNTTNT RR. A non-limiting representative non-structural NS5 sequence from which a subsequence or portion can be based upon is a sequence set forth as:

GTGNIGETLGEKWKSRLNALGKSEFQIYKKSGIQEVDRTLAKEGIKRGE
TDHHAVSRGSAKLRWFVERNMVTPEGKVVDLGCGRGGWSYYCGGLKNVR
EVKGLTKGGPGHEEPIPMSTYGWNLVRLQSGVDVFFTPPEKCDTLLCDI
GESSPNPTVEAGRTLRVLNLVENWLNNNTQFCIKVLNPYMPSVIEKMEA
LQRKYGGALVRNPLSRNSTHEMYWVSNASGNIVSSVNMISRMLINRFTM
RHKKATYEPDVDLGSGTRNIGIESEIPNLDIIGKRIEKIKQEHETSWHY
DQDHPYKTWAYHGSYETKQTGSASSMVNGVVRLLTKPWDVVPMVTQMAM
TDTTPFGQQRVFKEKVDTRTQEPKEGTKKLMKITAEWLWKELGKKKTPR
MCTREEFTRKVRSNAALGAIFTDENKWKSAREAVEDSRFWELVDKERNL
HLEGKCETCVYNMMGKREKKLGEFGKAKGSRAIWYMWLGARFLEFEALG
FLNEDHWFSRENSLSGVEGEGLHKLGYILRDVSKKEGGAMYADDTAGWD
TRITLEDLKNEEMVTNHMEGEHKKLAEAIFKLTYQNKVVRVQRPTPRGT
VMDIISRRDQRGSGQVGTYGLNTFTNMEAQLIRQMEGEGVFKSIQHLTV
TEEIAVQNWLARVGRERLSRMAISGDDCVVKPLDDRFASALTALNDMGK
VRKDIQQWEPSRGWNDWTQVPFCSHHFHELIMKDGRVLVVPCRNQDELI
GRARISQGAGWSLRETACLGKSYAQMWSLMYFHRRDLRLAANAICSAVP
SHWVPTSRTTWSIHAKHEWMTAEDMLTVWNRVWIQENPWMEDKTPVESW
EEIPYLGKREDQWCGSLIGLTSRATWAKNIQTAINQVRSLIGNEEYTDY
MPSMKRFRREEEEAGVLW.

In various embodiments, subsequences and portions of a structural or non-structural Dengue virus (DV) polypeptide sequence includes a T cell epitope that elicits an anti-DV CD8⁺ T cell response in vitro or in vivo. In particular aspects, the subsequence or portion of the Dengue virus (DV) polypeptide sequence elicits an anti-DV CD8⁺ T cell response, for example, induces, increases, enhances, stimulates or activates expression or production of a cytokine (e.g., IFN-gamma or TNF-alpha or IL-2), release of a cytotoxin (perforin or granulysin), or apoptosis of a target (e.g., DENV infected) cell, in vitro or in vivo.

Dengue virus (DV) peptides, subsequences and portions thereof, include amino acid substitutions, deletions or additions. Such substitutions, deletions or additions can be 1-2, 2-3, 3-4 or 5-6, 6-8, 7-10, 10-15, 15 to 20, 20-30, 30-50, 50-100, 100-150, or 150-200 amino acid residues. Substitutions include conservative, non-conservative, and conservative and non-conservative amino acid substitutions. In a particular embodiment, an amino acid substitution, deletion or addition is of a Dengue virus (DV) subsequence or portion that includes a T cell epitope, and elicits an anti-DV CD8⁺ T cell response in vitro or in vivo.

Dengue virus (DV) peptides, subsequences and portions thereof, can be included within larger amino acid sequences. Such larger sequences may include additional Dengue virus (DV) amino acid or protein sequences, such as one or more amino acid residues from a structural or non-structural protein sequence, provided that the region of the subsequence or portion that is identical to the structural or non-structural protein sequence is at least one amino acid less in length than the full length structural or non-structural protein sequence. Such larger sequences may include additional non-Dengue virus (DV) amino acid or protein sequences, such as one or more amino acid residues from another protein sequence to create a chimera or a fusion sequence. In various aspects, a Dengue virus (DV) portion or a subsequence can be within about a 5-10, 10-15, 15-20, 20-30, 30-50, 50-100, 100-150, 150-200, 200-250, 250-300, 300-400, or 400-500, amino acid residue sequence.

Dengue virus (DV) peptides, subsequences and portions thereof also provided include sequences with greater or less ability to function as a T cell epitope and elicit an anti-DV CD8⁺ T cell response in vitro or in vivo, than one or more of an exemplary core (C), membrane (M) or envelope (E) polypeptide, or an exemplary non-structural (NS) NS1, NS2A, NS2B, NS3, NS4A, NS4B or NS5 polypeptide.

The invention additionally provides nucleic acid sequences encoding Dengue virus (DV) peptides, subsequences and portions thereof. Such nucleic acid sequences include single or doublestrand nucleic acid (e.g., DNA, RNA), circular, and linear forms.

The invention further provides host cells expressing Dengue virus (DV) peptides, subsequences and portions thereof (e.g., nucleic acid sequences that encode Dengue virus (DV) peptide, subsequence or portion). Such host cells include prokaryotic, and eukaryotic (e.g., mammalian, such as human, plant, etc.) forms.

The invention moreover provides compositions (e.g., pharmaceutical) including Dengue virus (DV) peptides, subsequence and portions thereof, nucleic acid sequences encoding Dengue virus (DV) peptides, subsequences and portions thereof, and host cells expressing Dengue virus (DV) peptides, subsequences and portions thereof. A composition can be a liquid or a solid. In a particular aspect, a subsequence or portion of the Dengue virus (DV) polypeptide sequence includes a T cell epitope. Such pharmaceutical compositions can include an adjuvant (e.g., Freund's complete or incomplete adjuvant).

Kits that include Dengue virus (DV) peptides, subsequences and portions thereof, compositions, pharmaceutical formulations, nucleic acids, host cells, etc., are provided. Such kits optionally include instructions for treating (prophylactic or therapeutic), vaccinating or immunizing a subject against a Dengue virus (DV) infection, or treating (prophylactic or therapeutic) a subject having or at risk of having a Dengue virus (DV) infection or pathology.

In accordance with the invention, there are also provided methods of treating a subject for a Dengue virus (DV) infection (acute). In one embodiment, a method includes administering to a subject in need thereof an amount of a Dengue virus (DV) peptide, subsequence or portion thereof (e.g., a T cell epitope), to treat the subject for the pathogen infection.

In accordance with the invention, there are also provided prophylactic methods including methods of vaccinating and immunizing a subject against a Dengue virus (DV) infection (acute), for example, to protect a subject with protection against a Dengue virus (DV) infection (e.g., provide the subject with some protection against Dengue virus (DV) infection or pathology), to decrease or reduce the probability of a Dengue virus (DV) infection or pathology in a subject, to decrease or reduce susceptibility of a subject to a Dengue virus (DV) infection or pathology, or to inhibit or prevent a Dengue virus (DV) infection in a subject. In one embodiment, a method includes administering to a subject an amount of a Dengue virus (DV) peptide, subsequence or portion thereof (e.g., a T cell epitope), or a composition thereof (e.g., pharmaceutical formulation) prior to, substantially contemporaneously with or following exposure or contact of the subject with Dengue virus (DV), or exposure or contact of the subject to a subject (e.g., animal) infected with Dengue virus (DV), sufficient to vaccinate or immunize the subject against the Dengue virus (DV) infection or pathology. In various aspects, a method is sufficient to protect the subject from the Dengue virus (DV) infection (e.g., provide the subject with some protection against Dengue virus (DV) infection or pathology), to decrease or reduce the probability of Dengue virus (DV) infection in the subject, to decrease or reduce susceptibility of a subject to a Dengue virus (DV) infection or pathology.

Methods of the invention include administering a Dengue virus (DV) peptide, subsequence or portion thereof, or a composition thereof, at various times, frequencies and in various quantities. In particular embodiments, a Dengue virus (DV) peptide, subsequence or portion thereof, or a composition thereof, is administered prior to, substantially contemporaneously with or following contact, exposure to or infection with a Dengue virus (DV). In other embodiments, a Dengue virus (DV) peptide, subsequence or portion thereof, or a composition thereof, is administered prior to, substantially contemporaneously with or following exposure to, contact with or infection (acute) of the subject with a Dengue virus (DV). In additional embodiments, Dengue virus (DV) peptide, subsequence or portion thereof, or a composition thereof, is administered prior to, substantially contemporaneously with or following Dengue virus (DV) infection, development of a symptom associated with or caused by a Dengue virus (DV) (e.g., fever, rash, headache, pain behind the eyes, muscle or joint pain, nausea, vomiting, loss of appetite, etc.), Dengue virus (DV) replication or proliferation.

In accordance with the invention, there are also provided, there are further provided methods of inducing, increasing, promoting or stimulating anti-Dengue virus (DV) activity of CD8⁺ T cells in a subject. In one embodiment, a method includes administering to a subject an amount of a Dengue virus (DV) T cell epitope sufficient to induce, increase, promote or stimulate anti-Dengue virus (DV) activity of CD8⁺ T cells in the subject. In particular aspects, CD8⁺ T cells produce or express IFN gamma, TNF-alpha, or IL-2.

Methods of the invention also include increasing numbers or activation of an immune cell (e.g., CD4⁺ or CD8⁺ T cell) in in vitro or in vivo (e.g., in a subject with or at risk of a Dengue virus (DV) infection or pathology). In one embodiment, a method includes administering to a subject an amount of Dengue virus (DV) peptide, subsequence or portion thereof sufficient to increase numbers or activation of the immune cell in the subject. In another embodiment, a method includes administering to a subject an amount of Dengue virus (DV) peptide, subsequence or portion thereof, and administering a Dengue virus (DV) antigen, live or attenuated Dengue virus (DV), or a nucleic acid encoding all or a portion (e.g., an epitope) of any protein or proteinaceous Dengue virus (DV) antigen sufficient to increase numbers or activation of the immune cell in the subject. In particular aspects, the immune cell is a T cell, NKT cell, dendritic cell (DC), macrophage, neutrophil, eosinophil, mast cell, CD4⁺ or a CD8⁺ cell, CD14⁺, CD11b⁺ or CD11c⁺ cells.

Methods of the invention further include, among other things, increasing or inducing an anti-Dengue virus (DV) CD8⁺ or CD4⁺ T cell response in a subject with or at risk of a Dengue virus (DV) infection. In one embodiment, a method includes administering to a subject in need thereof an amount of Dengue virus (DV) peptide, subsequence or portion thereof sufficient to increase or induce an anti-Dengue virus (DV) CD8⁺ or CD4⁺ T cell response, including proliferation, cytokine secretion or cytotoxicity, or chemokine expression or production in the subject. In another embodiment, a method includes administering to a subject an amount of an Dengue virus (DV) peptide, subsequence or portion thereof, and administering a Dengue virus (DV) antigen, live or attenuated Dengue virus (DV), or a nucleic acid encoding all or a portion (e.g., an epitope) of any protein or proteinaceous Dengue virus (DV) antigen sufficient to increase or induce an anti-Dengue virus (DV) CD8⁺ or CD4⁺ T cell response, including proliferation, cytokine secretion or cytotoxicity, or chemokine expression or production in the subject.

Methods of the invention include, among other things, methods that provide a therapeutic or beneficial effect to a subject. In various non-limiting embodiments, a method decreases, reduces, inhibits, suppresses, controls or limits Dengue virus (DV) numbers or titer; decreases, reduces, inhibits, suppresses, prevents, controls or limits Dengue virus (DV) proliferation or replication; decreases, reduces, inhibits, suppresses, prevents, controls or limits the amount of a Dengue virus (DV) protein; or decreases, reduces, inhibits, suppresses, prevents, controls or limits the amount of a Dengue virus (DV) nucleic acid. In additional embodiments, a method increases, stimulates, enhances, promotes, augments or induces Dengue virus (DV) clearance or removal; increases, induces, enhances, augments, promotes or stimulates an immune response against a Dengue virus (DV); decreases, reduces, inhibits, suppresses, prevents, controls or limits Dengue virus (DV) pathology; decreases, reduces, inhibits, suppresses, prevents, controls or limits increases in Dengue virus (DV) numbers or titer; decreases, reduces, inhibits, suppresses, prevents, controls or limits increases in Dengue virus (DV) proliferation or replication, a Dengue virus (DV) protein, or a Dengue virus (DV) nucleic acid. In further embodiments, a method decreases, reduces, inhibits, suppresses, prevents, controls or limits transmission of Dengue virus (DV) (e.g., from a host (e.g., mosquito) to an uninfected subject or susceptible subject). In yet additional embodiments, a method decreases, reduces, inhibits, suppresses, prevents, controls, limits or improves one or more adverse (e.g., physical or physiological) symptoms, disorders, illnesses, diseases or complications associated with or caused by Dengue virus (DV) infection, or pathology. In still further embodiments, a method provides a subject with protection against a Dengue virus (DV) infection or pathology, or decreases, reduces, inhibits, or limits susceptibility or probability of a subject to a Dengue virus (DV) infection or pathology.

In various additional non-limiting embodiments, among other things, Dengue virus (DV) infection or pathoglogy is reduced, decreased, inhibited, limited, delayed or prevented, or a method decreases, reduces, inhibits, suppresses, prevents, controls or limits one or more adverse (e.g., physical or physiological) symptoms, disorders, illnesses, diseases or complications caused by or associated with Dengue virus (DV) infection, proliferation or replication, or pathology. In additional various non-limiting embodiments, a method reduces, decreases, inhibits, delays or prevents onset, progression, frequency, duration, severity, probability or susceptibility of one or more adverse symptoms, disorders, illnesses, diseases or complications caused by or associated with Dengue virus (DV) infection, proliferation or replication, or pathology. In further various non-limiting embodiments, a method accelerates, facilitates, enhances, augments, or hastens recovery of a subject from a Dengue virus (DV) infection or pathology, or one or more adverse symptoms, disorders, illnesses, diseases or complications caused by or associated with Dengue virus (DV) infection, proliferation or replication, or pathology. In yet additional non-limiting embodiments, a method stabilizes a Dengue virus (DV) infection, proliferation, replication, pathology, or an adverse symptom, disorder, illness, disease or complication caused by or associated with Dengue virus (DV) infection, proliferation or replication, or pathology, or decreases, reduces, inhibits, suppresses, prevents, limits or controls transmission of a Dengue virus (DV) from a host (mosquito) to an uninfected host (subject).

In additional various methods embodiments, Dengue virus (DV) peptide, subsequence or portion thereof, or a composition thereof, and a second active, such as a different Dengue virus (DV) peptide, subsequence or portion thereof, an anti-Dengue virus (DV) agent or a drug can be comined. Accordingly, combinations of a Dengue virus (DV) peptide, subsequence or portion thereof and a second active are provided.

In further embodiments, methods include administering to a subject, one or more times, a combination composition. In one embodiment, a Dengue virus (DV) peptide, subsequence or portion thereof, is administered as a combination composition with another Dengue virus (DV) peptide, subsequence or portion thereof, or an anti-Dengue virus (DV) agent or drug to a subject). In further various methods embodiments, an Dengue virus (DV) peptide, subsequence or portion thereof, and a second active, such as a different Dengue virus (DV) peptide, subsequence or portion thereof, an anti-viral agent or a drug are administered to a subject, one or more times, sequentially (e.g., Dengue virus (DV) peptide, subsequence or portion thereof, and an anti-viral agent or drug are administered separately to a subject, in a sequence). Second actives therefore include other Dengue virus (DV) peptide, subsequences and portions thereof, Dengue virus (DV) antigens, as well as immune enhancing antivirals, such as type I interferons.

Second actives, such as other Dengue virus (DV) antigens can therefore be included in the compositions and methods of the invention. For example, a Dengue virus (DV) antigen, live or attenuated Dengue virus (DV), or nucleic acid encoding all or a portion (e.g., an epitope) of a Dengue virus (DV) antigen can be included in a composition of the invention, or administered in a method of the invention. Such Dengue virus (DV) antigens can be from any serotype set forth herein or known to one of skill in the art, and can include an antigen that increases, stimulates, enhances, promotes, augments or induces a proinflammatory or adaptive immune response, numbers or activation of an immune cell (e.g., T cell, natural killer T (NKT) cell, dendritic cell (DC), macrophage, neutrophil, eosinophil, mast cell, CD4⁺ or a CD8⁺ cell, CD14⁺, CD11b⁺ or CD11c⁺ cells), an anti-Dengue virus (DV) CD4⁺ or CD8⁺ T cell response, production of a Th1 cytokine, or a T cell mediated immune response directed against a Dengue virus (DV). Thus, in further embodiments, Dengue virus (DV) peptide, subsequence or portion thereof, or a composition thereof, and another Dengue virus (DV) antigen, live or attenuated Dengue virus (DV), or nucleic acid encoding all or a portion (e.g., an epitope) of any protein or proteinaceous Dengue virus (DV) antigen are administered as a combination composition, or are administered separately, such as concurrently or sequentially, to a subject in order to effect treatment, vaccination or immunization, prior to, substantially contemporaneously with or following Dengue virus (DV) infection or pathology, or development of a symptom associated with or caused by a Dengue virus (DV) (e.g., fever, rash, headache, pain behind the eyes, muscle or joint pain, nausea, vomiting, loss of appetite, etc.). Methods of the invention therefore include administering to a subject an amount of Dengue virus (DV) peptide, subsequence or portion thereof, and administering a Dengue virus (DV) antigen, live or attenuated Dengue virus (DV), or a nucleic acid encoding all or a portion (e.g., an epitope) of any protein or proteinaceous Dengue virus (DV) antigen sufficient to increase production of a Th1 cytokine (e.g., interferon gamma, IL-2, TNF-alpha, etc.) in the subject.

### DESCRIPTION OF DRAWINGS

FIG. 1 shows the identification of DENV2-derived epitopes recognized by CD8⁺ T cells as determined by IFN-γ ELISPOT using CD8⁺ T cells isolated from C57BL/6 mice infected with 10¹¹ GE of the DENV2 strains PL046 (a clinical isolate) or S221 (mouse serum-passaged). The data are expressed as the mean number of net spot-forming cells (SFC) per 10⁶ CD8⁺ T cells. The 12 peptides shown were positive for both virus strains.

FIGS. 2A and 2B show confirmation of DENV2-derived CD8⁺ epitope identification by intracellular cytokine staining. A. IFN-γ production by DENV2-specific CD8⁺ T cells. The percent of CD8⁺ T cells producing IFN-γ is indicated. B. Summary of the DENV2-specific CD8⁺ IFN-γ⁺ response. Each symbol represents one mouse and the bar represents the geometric mean.

FIGS. 3A and 3B show CD8⁺ T cell activation induced by DENV2. A. The percent of CD8⁺ T cells expressing CD44 and CD62L from splenocytes harvested from naïve C57BL/6 mice or mice 7 days after infection with 10¹¹ GE of S221. B. The percent of DENV2-specific CD8⁺ T cells and the percent of CD44^{hi}, CD62^{lo}, and CD44^{hi}CD62L^{lo}CD8⁺ T cells. The data was taken from splenocytes stimulated with the eight major DENV2 peptides (individually; at 0.1 μg/ml), on day 7 after infection, in the presence of brefeldin A, and stained for surface CD8 and intracellular IFN-γ. The data are expressed as the mean percent±SEM of 11-14 mice tested in at least four independent experiments.

FIGS. 4A and 4B show the recognition of 8 major DENV2 CD8⁺ T cell epitopes in IFN-α/βR^{-/-} mice. The data was taken from splenocytes harvested from IFN-α/βR^{-/-} mice 7 days after infection with 10¹⁰ GE S221 which were re-stimulated in vitro with individual DENV2 peptides (or an irrelevant peptide) at 0.1 μg/ml in the presence of brefeldin A for 5 h; and then stained for CD8 and intracellular IFN-γ. A. The percent of CD8⁺ T cells producing IFN-γ. B. Summary of the DENV2-specific CD8⁺ IFN-γ⁺ response. Each symbol represents one mouse and the bar represents the geometric mean.

FIGS. 5A to 5F show DENV infection results in a CD8⁺ T cell response in wild-type and IFN-α/βR^{-/-} mice, but detectable levels of viremia only in IFN-α/βR^{-/-} mice. A. The DENV RNA levels in the serum measured by real-time RT-PCR from bled Wild-type mice (n=3) infected with 10¹¹ GE of S221 and IFN-α/βR^{-/-} mice (n=6) infected with 10¹⁰ GE. The dashed line indicates the limit of detection. B. The percentage of CD44^{hi}CD62L^{lo} cells (gated on CD8⁺ T cells) determined from blood lymphocytes obtained from wild-type mice (n=4) on days 3, 6, 8, and 13 after infection with 10¹¹ GE of S221. The percentage of CD44^{hi}CD62L^{lo} cells (gated on CD8⁺ T cells) is indicated. C. The numbers of splenic CD8⁺ T cells in naïve IFN-α/βR^{-/-} mice (n=4) and IFN-α/βR^{-/-} mice infected with 10¹⁰ GE of S221 (n=7). *** p<0.0001 for naïve versus infected mice. D. The percentage of CD44^{hi}CD62L^{lo} cells (gated on CD8⁺ T cells) determined from blood lymphocytes obtained from IFN-α/βR^{-/-} mice (n=3) on days 3, 5, 7, 10, and 13 after infection with 10¹⁰ GE of S221. The percentage of CD44^{hi}CD62L^{lo} cells (gated on CD8⁺ cells) is shown. E. Expression of CD44 and CD62L on CD8⁺ T cells from naïve and infected IFN-α/βR^{-/-} mice as determined by splenocytes harvested from naïve IFN-α/βR^{-/-} mice or mice 7 days after infection with 10¹⁰ GE of S221. F. The percent of DENV2-specific CD8⁺ T cells and the percent of CD44^{hi}, CD62^{lo}, and CD44^{hi}CD62L^{lo} CD8⁺ T cells from naïve or infected IFN-α/βR^{-/-} mice as determined from splenocytes harvested from naïve IFN-α/βR^{-/-} mice or mice 7 days after infection with 10¹⁰ GE of S221. The data are expressed as the percent±SEM of seven mice tested in three independent experiments.

FIGS. 6A to 6D show depletion of CD8⁺ T cells prior to DENV infection results in increased viral loads as determined from IFN-α/βR^{-/-} mice depleted of CD8⁺ T cells by administration of an anti-CD8 Ab (or given an isotype control Ab) 3 days and 1 day before infection with 10¹¹ GE of S221 and sacrificed 6 days later. The DENV RNA levels in the serum, spleen, liver and brain were quantified by real-time RT-PCR. Data are expressed as DENV copies per ml of sera, or DENV units normalized to 18S rRNA levels for the spleen, liver, and brain. A. Serum. B. Spleen. C. Liver. D. Brain. Each symbol represents one mouse, the bar represents the geometric mean, and the dashed line is the limit of detection. ** p<0.001 for serum, *** p<0.0001 for spleen and brain; and p=0.39 for viral load in the liver of CD8-depleted mice compared with control mice.

FIGS. 7A to 7E show depletion of CD4⁺ and/or CD8⁺ T cells prior to DENV infection results in increased viral loads. IFN-α/βR^{-/-} mice were depleted of CD4⁺ and/or CD8⁺ T cells by i.p. administration of anti-CD4 Ab (GK1.5, 250 μg) and/or anti-CD8 Ab (2.43, 250 μg) or given an isotype control Ab 3 days and 1 day before i.v. infection with 10¹¹ GE of the DENV2 strain, S221. Mice were sacrificed 6 days later, and DENV RNA levels in the serum, spleen, kidney, small intestine and brain were quantified by real-time RT-PCR. Data are expressed as DENV copies per ml of sera, or DENV units normalized to 18S rRNA levels for the tissues. A. Serum. B. Spleen C. Kidney D. Brain. E. Small intestine. Each symbol represents one mouse, the bar represents the geometric mean, and the dashed line is the limit of detection.

FIGS. 8A to 8D show confirmation of DENV-derived CD8⁺ T cell epitope identification in wild-type and IFN-α/βR^{-/-} mice by ICS. A. and B. The number of CD8⁺ T cells producing IFN-γ from wild-type and 7 IFN-α/βR^{-/-} mice as indicated. Splenocytes were harvested from wild-type (A) or IFN-α/βR^{-/-} (B) mice 7 days after infection with 10¹¹ or 10¹⁰ GE of S221, respectively, were re-stimulated in vitro with individual DENV peptides or an irrelevant peptide. Cells were then stained for surface CD8 and intracellular IFN-γ and analyzed by flow cytometry. The response to the irrelevant peptide was subtracted from the response to each DENV peptide, and the number of CD8⁺ T cells producing IFN-γ is indicated. Results are expressed as the mean±SEM of 13 wild-type and 7 IFN-α/βR^{-/-} mice tested in at least three independent experiments. C and D. Kinetics of the DENV-specific CD8⁺ T cell response. Wild-type mice ((C), n=4) and IFN-α/βR^{-/-} mice ((D), n=3) were infected with 10¹¹ or 10¹⁰ GE of S221, respectively, and blood lymphocytes were isolated at various time points. Stimulation and ICS were performed as in (A) and (B), and the percentage of CD8⁺ T cells producing IFN-γ is shown.

FIG. 9 shows DENV-specific CD8⁺ T cells have a polyfunctional phenotype. Splenocytes harvested from wild-type or IFN-α/βR^{-/-} mice 7 days after infection with 10¹¹ or 10¹⁰ GE of S221, respectively, were re-stimulated in vitro with C 51-59, NS4B 99-107, or an irrelevant peptide. An anti-CD107a Ab was added for the duration of the stimulation. Cells were then stained for surface CD8, intracellular IFN-γ and TNF-α, and analyzed by flow cytometry. Representative density plots are shown.

FIGS. 10A and 10B show DENV-specific CD8⁺ T cells are cytotoxic and protective in vivo. A. In vivo killing of DENV peptide-pulsed cells. IFN-α/βR^{-/-} mice infected 7 days previously with 10¹⁰ GE of S221 were injected i.v. with CFSE-labeled target cells pulsed with C 51-9, NS2A 8-15, NS4B 99-107, NS5 237-245 or a pool of the four peptides (n=3-6 mice per group). After 4 h, splenocytes were harvested, analyzed by flow cytometry, and the percentage killing was calculated. B. Peptide immunization results in enhanced DENV clearance. IFN-α/βR^{-/-} mice were immunized s.c. with 50 μg each of four DENV peptides (C 51-59, NS2A 8-15, NS4B 99-107, NS5 237-245) and 100 μg helper peptide in IFA, or mock-immunized with 100 !Az helper peptide and DMSO in IFA. Mice were infected with 10¹¹ GE of S221 12-13 days later, and then sacrificed 4 days after infection. A separate group of peptide-immunized mice were depleted of CD8⁺ T cells prior to infection. DENV RNA levels in the serum were quantified by real-time RT-PCR. Each symbol represents one mouse; the bar represents the geometric mean, and the dashed line the limit of detection. *** p<0.0001 comparing peptide-immunized with mock-immunized mice; ** p<0.001 comparing peptide-immunized with peptide-immunized/CD8-depleted mice.

FIG. 11 shows identification of DENV2-derived human HLA A*0201 epitopes by IFN-γ ELISPOT. The proteome of the DENV2 strain, S221, was inspected for the presence of peptides predicted to bind HLA A*0201 with high affinity. A total of 68 potential H-2^{b} binding peptides were identified. HLA A*0201 transgenic mice were infected i.v. with 10¹¹ GE of the DENV2 strain, S221. Seven days post-infection, splenocytes were harvested and CD8⁺ T cells isolated. CD8⁺ T cells (1.75×10⁵) were stimulated with HLA-A*0201-restricted Jurkat cells and 1 μg/ml of individual S221-derived A*02-predicted binding peptides, and IFN-γ ELISPOT was performed. The data are expressed as the mean number of net spot-forming cells (SFC) per 10⁶ CD8⁺ T cells. The top 20 predicted epitopes, which includes the two positive peptides identified (indicated with an asterisk), are shown. The criteria for positivity were a stimulation index of ≧2.0, p<0.05 when compared with an irrelevant control peptide, and net SFC/10⁶ cells of ≧20.

### DETAILED DESCRIPTION

The invention is based at least in part on Dengue virus (DV) peptides, subsequences and portions thereof. Invention Dengue virus (DV) peptides, subsequences and portions thereof, including T cell epitopes that can elicit (produce, induce, increase, enhance, stimulate or activate) an anti-DV CD8+ T cell response in vitro or in vivo, are useful in treatment, vaccination and immunization methods. For example, invention Dengue virus (DV) peptide, subsequence or portion thereof, are useful in methods of treating a subject for having or at risk of having Dengue virus (DV) infection or pathology.

Dengue virus (DV) peptide, subsequences and portions thereof include T cell epitopes. A T cell epitope can elicit (produce, induce, increase, enhance, stimulate or activate) an anti-DV CD8⁺ T cell response in vitro or in vivo. Exemplary T cell epitopes can include or consist of a subsequence or portion of Dengue virus (DV) structural Core, Membrane or Envelope polypeptide sequence, or a subsequence or portion of a Dengue virus (DV) non-structural (NS) NS1, NS2A, NS2B, NS3, NS4A, NS4B or NS5 polypeptide sequence. Specific non-limiting examples of Dengue virus (DV) structural protein include or consist of a sequence set forth as: GMLQGRGPL (SEQ ID NO:1); VAFLRFLTI (SEQ ID NO:2); RALIFILL (SEQ ID NO:3); MTMRCIGI (SEQ ID NO:4); VSWTMKIL (SEQ ID NO:5); or RLITVNPIV (SEQ ID NO:13), or a subsequence thereof or an amino acid substitution thereof. Specific non-limiting examples of Dengue virus (DV) non-structural (NS) protein include or consist of a sequence set forth as: FSLGVLGM (SEQ ID NO:6); VAVSFVTLI (SEQ ID NO:7); LAVTIMAIL (SEQ ID NO:8); TAIANQATV (SEQ ID NO:9); TAIANQATV (SEQ ID NO:10); YSQVNPITL (SEQ ID NO:11); RMLINRFTM (SEQ ID NO:12); or KLAEAIFKL (SEQ ID NO:14), a subsequence thereof or an amino acid substitution thereof.

Additional Dengue virus (DV) peptide, subsequences and portions thereof can be based upon or derived from DENV serotypes, such as DENV1, DENV2, DENV3 or DENV4 serotypes. A subsequence or portion of a Dengue virus (DV) non-structural (NS) NS1, NS2A, NS2B, NS3, NS4A, NS4B or NS5 polypeptide, or structural core (C), membrane (M) or envelope (E) polypeptide, can be a sequence having 75% or more (e.g., 80%, 85%, 90%, 95%, 96%, 97%, 98%, 99%, 100%) identity to a non-structural (NS) or structural polypeptide of a Dengue virus (DV) serotype, such as a DENV1, DENV2, DENV3 or DENV4 serotype.

Thus, in accordance with the invention, there are also provided Dengue virus (DV) peptides, subsequences and portions thereof that exhibit sequence identity to a reference Dengue virus (DV) peptide, subsequence or portion thereof. In one embodiment, an Dengue virus (DV) peptide, subsequences and portions thereof includes or consists of a sequence at least 60% or more (e.g., 65%, 70%, 75%, 80%, 85%, 90%, 95%, etc.) identical to any Dengue virus (DV) peptide, subsequence or portion thereof set forth herein (e.g., SEQ ID NOs:1 to 14).

In another embodiment, a Dengue virus (DV) peptide, subsequences and portions thereof includes or consist of a Dengue virus (DV) peptide, subsequence or portion thereof set forth as SEQ ID NOs:1 to 14, wherein the⁻Dengue virus (DV) peptide, subsequence or portion thereof has one or more amino acid additions, deletions or substitutions of any of SEQ ID NOs:1 to 14. In particular aspects, a sequence is at least 80% or more, e.g., 80-85%, 85-90%, 90-95%, 95-100% identical to Dengue virus (DV) peptide, subsequence or portion thereof set forth as any of SEQ ID NOs:1 to 14.

T cell epitopes typically are short amino acid sequences, e.g. about five to 15 amino acids in length. Linear or contiguous T cell epitopes include a continuous amino acid sequence, such as a 5 to 15 amino acid sequence, which can elicit an anti-DV CD8⁺ T cell response in vitro or in vivo.

A non-limiting example of a subsequence or portion of a Dengue virus (DV) polypeptide sequence includes or consists of a subsequence or portion of Dengue virus (DV) structural Core, Membrane or Envelope polypeptide sequence. A more particular non-limiting example of Dengue virus (DV) structural protein include or consist of a sequence set forth as: GMLQGRGPL (SEQ ID NO:1); VAFLRFLTI (SEQ ID NO:2); RALIFILL (SEQ ID NO:3); MTMRCIGI (SEQ ID NO:4); VSWTMKIL (SEQ ID NO:5); or RLITVNPIV (SEQ ID NO:13), or a subsequence thereof or an amino acid substitution thereof.

A non-limiting example of a subsequence or portion of a Dengue virus (DV) polypeptide sequence includes or consists of a subsequence or portion of Dengue virus (DV) non-structural (NS) NS1, NS2A, NS2B, NS3, NS4A, NS4B or NS5 polypeptide sequence. A more particular non-limiting example of Dengue virus (DV) non-structural (NS) protein include or consist of a sequence set forth as: FSLGVLGM (SEQ ID NO:6); VAVSFVTLI (SEQ ID NO:7); LAVTIMAIL (SEQ ID NO:8); TAIANQATV (SEQ ID NO:9); TAIANQATV (SEQ ID NO:10); YSQVNPITL (SEQ ID NO:11); RMLINRFTM (SEQ ID NO:12); or KLAEAIFKL (SEQ ID NO:14), a subsequence thereof or an amino acid substitution thereof.

A non-limiting Core sequence from which a subsequence or portion can be based upon is a sequence set forth as: MNNQRKKARNTPFNMLKRERNRVSTVQQLTKRFSEGMLQGRGPLKLFMALVAFLRFLTIPP TAGILKRWGTIKKSKAINVLRGFRKEIGRMLNILNRRRRTAGMIIMLIPTVMA. A non-limiting Membrane sequence from which a subsequence or portion can be based upon is a sequence set forth as: FHLTTRNGEPHMIVSRQEKGKSLLFKTGDGVNMCTLMAMDLGELCEDTITYKCPLLRQNEP EDIDCWCNSTSTWVTYGTCITTGEHRREKRSVALVPHVGMGLETRTETWMSSEGAWKIIA QRIETWILRHPGFTIMAAILAYTIGTTHFQRALIFILLTAVAPSMT. A non-limiting Envelope sequence from which a subsequence or portion can be based upon is a sequence set forth as: MRCIGISNRINVEGVSGGSWVDIVLEHGSCVTTMAKNKPTLDFELIKTEAKQSATLRKYCIE AKLTNTTTESRCPTQGEPSLNEEQDKRFVCKHSMVDRGWGNGCGLFGKGGIVTCAMFTCK KNMKGKVVQPENLEYTIVITPHSGEEHAVGNDTGKHGKEIKITPQSSITEAELTGYGTVTME CSPRTGLDFNEMVLLQMENKAWLVHRQWFLDLPLPWLPGADTQGSNWIQKETLVTFKNP HAKKQDVVVLGSQEGAMHTALTGATEIQMSSGNLLFTGHLKCRLRMDKLQLKGMSYSMC TGKITKVVKEIAETQHGTIVIRVQYEGDGSPCKIPFEIMDLEKRHVLGRLITVNPIVTEKDSPV NIEAEPPFGDSYIIIGVEPGQLKLNWFKKGSSIGQMLETTMRGAKRMAILGDTAWDFGSLGG VFTSIGKALHQVFGAIYGAAFSGVSWTMKILIGVIITWIGMNSRSTSLSVSLVLVGVVTLYLG VMVQA. A non-limiting Envelope sequence from which a subsequence or portion can be based upon is an Envelope sequence with a substitution of E₁₂₄ N→D; or E₁₂₈ K→E.

A non-limiting non-structural NS1 sequence from which a subsequence or portion can be based upon is a sequence set forth as: ADSGCVVSWKNKELKCGSGIFITDNVHTWTEQYKFQPESPSKLASAIQKAHEEGICGIRSVT RLENLMWKQITPELNHILSENEVKLTIMTGDIKGIMQAGKRSLRPQPTELKYSWKTWGKAK MLSTESEINQTFLIDGPETAECPNTNRAWNSLEVEDYGEGVFTTNIWLKLREKQDVFCDSKL MSAAIKDNRAVHADMGYWIESALNDTWKIEKASFIEVKSCHWPKSHTLWSNEVLESEMIIP KNFAGPVSQHNYRPGYHTQTAGPWHLGKLEMDFDFCEGTTVVVTEDCGNRGPSLRTTTAS GKLITEWCCRSCTLPPLRYRGEDGCWYGMEIRPLKEKEENLVNSLVTA. A non-limiting non-structural NS2A sequence from which a subsequence or portion can be based upon is a sequence set forth as: GHGQIDNFSLGVLGMALFLEEMLRTRVGTKHAILLVAVSFVTLITGNMSFRDLGRVMVMV GATMTDDIGMGVTYLALLAAFKVRPTFAAGLLLRKLTSKELMMTTIGIVLLSQSTIPETILEL TDALALGMMVLKMVRKMEKYQLAVTIMAILCVPNAVILQNAWKVSCTILAVVSVSPLFLT SSQQKADWIPLALTIKGLNPTAIFLTTLSRTNKKR. A non-limiting non-structural NS2B sequence from which a subsequence or portion can be based upon is a sequence set forth as: SWPLNEAIMAVGMVSILASSLLKNDIPMTGPLVAGGLLTVCYVLTGRSADLELERAADVK WEDQAEISGSSPILSITISEDGSMSIKNEEEEQTLTILIRTGLLVISGLFPVSLPITAAAWYLWEV KKQR. A non-limiting non-structural NS3 sequence from which a subsequence or portion can be based upon is a sequence set forth as: AGVLWDVPSPPPVGKAELEDGAYRIKQKGILGYSQIGAGVYKEGTFHTMWHVTRGAVLM HKGKRIEPSWADVKKDLISYGGGWKLEGEWKEGEEVQVLALEPGKNPRAVQTKPGLEKTN AGTIGAVSLDFSPGTSGSPIIDKKGKVVGLYGNGVVTRSGAYVSAIAQTEKSIEDNPEIEDDIF RKRKLTIMDLHPGAGKTKRYLPAIVREAIKRGLRTLILAPTRVVAAEMEEALRGLPIRYQTP AIRAEHTGREIVDLMCHATFTMRLLSPVRVPNYNLIIMDEAHFTDPASIAARGYISTRVEMG EAAGIFMTATPPGSRDPFPQSNAPIMDEEREIPERSWSSGHEWVTDFKGKTVWFVPSIKAGN DIAACLRKNGKKVIQLSRKTFDSEYVKTRTNDWDEVVTTDISEMGANFKAERVIDPRRCMK PVILTDGEERVILAGPMPVTHSSAAQRRGRIGRNPKNENDQYIYMGEPLENDEDCAHWKEA KMLLDNINTPEGIIPSMEEPEREKVDAIDGEYRLRGEARKTFVDLMRRGDLPVWLAYRVAA EGINYADRRWCEDGIKNNQILEENVEVEIWTKEGERKKLKPRWLDARIYSDPLALKEFKEFA AGRK. A non-limiting non-structural NS4A sequence from which a subsequence or portion can be based upon is a sequence set forth as: SLTLSLITEMGRLPTFMTQKARDALDNLAVLHTAEAGGRAYNHALSELPETLETLLLLTLLA TVTGGIFLFLMSGRGIGKMTLGMCCIITASILLWYAQIQPHWIAASIILEFFLIVLLIPEPEKQR TPQDNQLTYVVIAILTVVAATMA. A non-limiting non-structural NS4B sequence from which a subsequence or portion can be based upon is a sequence set forth as: NEMGFLEKTKKDLGLGSITTQQPESNILDIDLRPASAWTLYAVATTFVTPMLRHSIENSSVN VSL [symbol] LMGLGKGWPLSKMDIGVPLLAIGCYSQVNPITLTAALFLLVAHYAIIGPG LQAKATREAQKRAAAGIMKNPTVDGITVIDLDPIPYDPKFEKQLGQVMLLVLCVTQVLMM RTTWALCEALTLATGPISTLWEGNPGREWNTTIAVSMANIFRGSYLAGAGLLFSIMKNTTNT RR. A non-limiting non-structural NS5 sequence from which a subsequence or portion can be based upon is a sequence set forth as:

GTGNIGETLGEKWKSRLNALGKSEFQIYKKSGIQEVDRTLAKEGIKRGE
TDHHAVSRGSAKLRWFVERNMVTPEGKVVDLGCGRGGWSYYCGGLKNVR
EVKGLTKGGPGHEEPIPMSTYGWNLVRLQSGVDVFFTPPEKCDTLLCDI
GESSPNPTVEAGRTLRVLNLVENWLNNNTQFCIKVLNPYMPSVIEKMEA
LQRKYGGALVRNPLSRNSTHEMYWVSNASGNIVSSVNMISRMLINRFTM
RHKKATYEPDVDLGSGTRNIGIESEIPNLDIIGKRIEKIKQEHETSWHY
DQDHPYKTWAYHGSYETKQTGSASSMVNGVVRLLTKPWDVVPMVTQMAM
TDTTPFGQQRVFKEKVDTRTQEPKEGTKKLMKITAEWLWKELGKKKTPR
MCTREEFTRKVRSNAALGAIFTDENKWKSAREAVEDSRFWELVDKERNL
HLEGKCETCVYNMMGKREKKLGEFGKAKGSRAIWYMWLGARFLEFEALG
ELNEDHWFSRENSLSGVEGEGLHKLGYILRDVSKKEGGAMYADDTAGWD
TRITLEDLKNEEMVTNHMEGEHKKLAEAIFKLTYQNKVVRVQRPTPRGT
VMDIISRRDQRGSGQVGTYGLNTFTNMEAQLIRQMEGEGVFKSIQHLTV
TEEIAVQNWLARVGRERLSRMAISGDDCVVKPLDDRFASALTALNDMGK
VRKDIQQWEPSRGWNDWTQVPFCSHHFHELIMKDGRVLVVPCRNQDELI
GRARISQGAGWSLRETACLGKSYAQMWSLMYFHRRDLRLAANAICSAVP
SHWVPTSRTTWSIHAKHEWMTAEDMLTVWNRVWIQENPWMEDKTPVESW
EEIPYLGKREDQWCGSLIGLTSRATWAKNIQTAINQVRSLIGNEEYTDY
MPSMKRFRREEEEAGVLW.

The invention provides isolated Dengue virus (DV) peptides, including or consisting of a subsequence or portion of a structural core (C), membrane (M) or envelope (E) polypeptide sequence, or a non-structural (NS) NS1, NS2A, NS2B, NS3, NS4A, NS4B or NS5 polypeptide sequence. In particular embodiments, an isolated subsequence or portion of the Dengue virus (DV) polypeptide sequence includes a T cell epitope.

The term “isolated,” when used as a modifier of a composition (e.g., Dengue virus (DV) peptides, subsequences and portions thereof, nucleic acids encoding same, etc.), means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man.

An “isolated” composition (e.g., Dengue virus (DV) peptide, subsequence or portion thereof) can also be “substantially pure” or “purified” when free of most or all of the materials with which it typically associates with in nature. Thus, an isolated Dengue virus (DV) peptide, subsequence or portion thereof, that also is substantially pure or purified does not include polypeptides or polynucleotides present among millions of other sequences, such as peptides of an peptide library or nucleic acids in a genomic or cDNA library, for example. A “substantially pure” or “purified” composition can be combined with one or more other molecules. Thus, “substantially pure” or “purified” does not exclude combinations of compositions, such as combinations of Dengue virus (DV) peptides, subsequences and portions thereof, and other antigens, agents, drugs or therapies.

The term “chimeric” and grammatical variations thereof, when used in reference to a sequence, means that the amino acid sequence contains one or more portions that are derived from, obtained or isolated from, or based upon two or more different proteins. For example, a portion of the sequence may be a Dengue virus (DV) peptide, subsequence or portion, and another portion of the sequence may be from a different Dengue virus (DV) peptide sequence, or a non-Dengue virus (DV) sequence.

Dengue virus (DV) peptides, subsequences and portions thereof of the invention include those having at least partial sequence identity to one or more exemplary Dengue virus (DV) peptides, subsequences and portions thereof (e.g., SEQ ID NOs:1 to 14). The percent identity of such sequences can be as little as 60%, or can be greater (e.g., 65%, 70%, 75%, 80%, 85%, 90%, 95%, 96%, 97%, 98%, 99%, etc.). The percent identity can extend over the entire sequence length or a portion of the sequence. In particular aspects, the length of the sequence sharing the percent identity is 2, 3, 4, 5 or more contiguous amino acids, e.g., 6, 7, 8, 9, 10, 11, 12, 13, 14, 15, 16, 17, 18, 19, 20, etc. contiguous amino acids. In additional particular aspects, the length of the sequence sharing the percent identity is 20 or more contiguous amino acids, e.g., 20, 21, 22, 23, 24, 25, 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet further particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-110, etc. contiguous amino acids.

The term “identity” and grammatical variations thereof, mean that two or more referenced entities are the same. Thus, where two Dengue virus (DV) peptides, subsequences or portions thereof are identical, they have the same amino acid sequence. The identity can be over a defined area (region or domain) of the sequence. “Areas, regions or domains” of homology or identity mean that a portion of two or more referenced entities share homology or are the same.

The extent of identity between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch −2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

In accordance with the invention, there are provided Dengue virus (DV) peptides, subsequences and portions thereof that include modified and variant forms. As used herein, the terms “modify” or “variant” and grammatical variations thereof, mean that a Dengue virus (DV) peptide, subsequence or portion thereof deviates from a reference sequence (e.g., any of SEQ ID NOS:1 to 14). Modified and variant Dengue virus (DV) peptides, subsequences and portions thereof may therefore have greater or less activity or function than a reference Dengue virus (DV) peptide, subsequence or portion thereof, but at least retain partial activity or function of the reference sequence (e.g., any of SEQ ID NOs:1 to 14). Thus, Dengue virus (DV) peptides, subsequences and portions thereof include sequences having substantially the same, greater or less relative activity or function as a T cell epitope than a reference T cell epitope (e.g., any of SEQ ID NOs:1 to 14), for example, an ability to elicit (produce, induce, increase, enhance, stimulate or activate) an anti-DV CD8⁺ T cell response in vitro or in vivo.

Non-limiting examples of modifications include one or more amino acid substitutions (e.g., 1-3, 3-5, 5-10, 10-15, 15-20, 20-25, or more residues), additions (e.g., insertions or 1-3, 3-5, 5-10, 10-15, 15-20, 20-25, or more residues) and deletions (e.g., subsequences or fragments) of a reference Dengue virus (DV) peptide, subsequence or portion thereof. In particular embodiments, a modified or variant sequence retains at least part of a function or an activity of unmodified sequence. Such modified forms and variants can have less than, the same, or greater, but at least a part of, a function or activity of a reference sequence, for example, the ability to elicit (produce, induce, increase, enhance, stimulate or activate) an anti-DV CD8⁺ T cell response in vitro or in vivo. CD8⁺ T cell responses elicited include, for example induced, increased, enhanced, stimulate or activate expression or production of a cytokine (e.g., IFN-gamma), release of a cytotoxin (perforin or granulysin), or apoptosis of a target (e.g., DENV infected) cell.

Specific non-limiting examples of substitutions include conservative and non-conservative amino acid substitutions. A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

An addition can be the covalent or non-covalent attachment of any type of molecule to the sequence. Specific examples of additions include glycosylation, acetylation, phosphorylation, amidation, formylation, ubiquitination, and derivatization by protecting/blocking groups and any of numerous chemical modifications. Additional specific non-limiting examples of an addition is one or more additional amino acid residues. In particular embodiments, an addition is a fusion (chimeric) sequence, an amino acid sequence having one or more molecules not normally present in a reference native (wild type) sequence covalently attached to the sequence. A particular example is an amino acid sequence of another sequence to produce a chimera.

Another particular example of a modified sequence having an amino acid addition is one in which a second heterologous sequence, i.e., heterologous functional domain is attached (covalent or non-covalent binding) that confers a distinct or complementary function. Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral), a metal (gold, silver), radioisotope. For example, a tag such as T7 or polyhistidine can be attached in order to facilitate purification or detection of a T cell epitope. Thus, in other embodiments the invention provides Dengue virus (DV) peptides, subsequences and portions thereof and a heterologous domain, wherein the domain confers a distinct function, i.e. a heterologous functional domain, on the Dengue virus (DV) peptides, subsequences and portions thereof.

Further non-limiting examples of additions are detectable labels. Thus, in another embodiment, the invention provides Dengue virus (DV) peptides, subsequences and portions thereof that are detectably labeled. Specific examples of detectable labels include fluorophores, chromophores, radioactive isotopes (e.g., S³⁵, P³², I¹²⁵), electron-dense reagents, enzymes, ligands and receptors. Enzymes are typically detected by their activity. For example, horseradish peroxidase is usually detected by its ability to convert a substrate such as 3,3-′,5,5-′-tetramethylbenzidine (TMB) to a blue pigment, which can be quantified.

Linkers, such as amino acid or peptidimimetic sequences may be inserted between the sequence and the addition (e.g., heterologous functional domain) so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary without significantly affecting a function or activity of the fusion protein (see, e.g., U.S. Pat. No. 6,087,329). Linkers further include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

Another non-limiting example of an addition is an insertion of an amino acid within any sequence of Dengue virus (DV) peptides, subsequences and portions thereof (e.g., any of SEQ ID NOS:1 to 14). In particular embodiments, an insertion is of one or more amino acid residues in any of a Dengue virus (DV) peptide, subsequence or portion thereof, such as any of SEQ ID NOs: 1 to 14.

Modified Dengue virus (DV) peptides, subsequences and portions thereof also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond. Dengue virus (DV) peptides, subsequences and portions thereof may be modified in vitro or in vivo, e.g., post-translationally modified to include, for example, sugar residues, phosphate groups, ubiquitin, fatty acids, lipids, etc.

Dengue virus (DV) peptides, subsequences and portions thereof including modified forms can be produced by any of a variety of standard protein purification or recombinant expression techniques. For example, a Dengue virus (DV) peptide, subsequence or portion thereof can be produced by standard peptide synthesis techniques, such as solid-phase synthesis. A portion of the protein may contain an amino acid sequence such as a T7 tag or polyhistidine sequence to facilitate purification of expressed or synthesized protein. The protein may be expressed in a cell and purified. The protein may be expressed as a part of a larger protein (e.g., a fusion or chimera) by recombinant methods.

Dengue virus (DV) peptides, subsequences and portions thereof including modified forms can be made using recombinant DNA technology via cell expression or in vitro translation. Polypeptide sequences including modified forms can also be produced by chemical synthesis using methods known in the art, for example, an automated peptide synthesis apparatus (see, e.g., Applied Biosystems, Foster City, Calif.).

The invention also provides nucleic acids encoding Dengue virus (DV) peptides, subsequences and portions thereof. Such nucleic acid sequences encode a sequence at least 60% or more (e.g., 65%, 70%, 75%, 80%, 85%, 90%, 95%, etc.) identical to a Dengue virus (DV) peptide, subsequence or portion thereof. In an additional embodiment, a nucleic acid encodes a sequence having one or more amino acid additions (insertions), deletions or substitutions of a Dengue virus (DV) peptide, subsequences or portion thereof, such as any of SEQ ID NOs:1 to 14.

The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleotides. Nucleic acids include single, double or triplex, circular or linear, molecules. Exemplary nucleic acids include but are not limited to: RNA, DNA, cDNA, genomic nucleic acid, naturally occurring and non naturally occurring nucleic acid, e.g., synthetic nucleic acid.

Nucleic acids can be of various lengths. Nucleic acid lengths typically range from about 20 nucleotides to 20 Kb, or any numerical value or range within or encompassing such lengths, 10 nucleotides to 10Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. In particular aspects, a nucleic acid sequence has a length from about 10-20, 20-30, 30-50, 50-100, 100-150, 150-200, 200-250, 250-300, 300-400, 400-500, 500-1000, 1000-2000, nucleotides, or any numerical value or range within or encompassing such lengths. Shorter polynucleotides are commonly referred to as “oligonucleotides” or “probes” of single- or double-stranded DNA. However, there is no upper limit to the length of such oligonucleotides.

Nucleic acid sequences further include nucleotide and nucleoside substitutions, additions and deletions, as well as derivatized forms and fusion/chimeric sequences (e.g., encoding recombinant polypeptide). For example, due to the degeneracy of the genetic code, nucleic acids include sequences and subsequences degenerate with respect to nucleic acids that encode Dengue virus (DV) peptides, subsequences and portions thereof, as well as variants and modifications thereof (e.g., substitutions, additions insertions and deletions).

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to the encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as eukaryote or mammalian cell, yeast or bacteria, in an animal or in a plant).

Nucleic acid may be inserted into a nucleic acid construct in which expression of the nucleic acid is influenced or regulated by an “expression control element.” An “expression control element” refers to a nucleic acid sequence element that regulates or influences expression of a nucleic acid sequence to which it is operatively linked. Expression control elements include, as appropriate, promoters, enhancers, transcription terminators, gene silencers, a start codon (e g., ATG) in front of a protein-encoding gene, etc.

An expression control element operatively linked to a nucleic acid sequence controls transcription and, as appropriate, translation of the nucleic acid sequence. Expression control elements include elements that activate transcription constitutively, that are inducible (i e., require an external signal for activation), or derepressible (i.e., require a signal to turn transcription off; when the signal is no longer present, transcription is activated or “derepressed”), or specific for cell-types or tissues (i.e., tissue-specific control elements).

Nucleic acid may be inserted into a plasmid for propagation into a host cell and for subsequent genetic manipulation. A plasmid is a nucleic acid that can be propagated in a host cell, plasmids may optionally contain expression control elements in order to drive expression of the nucleic acid encoding Dengue virus (DV) peptides, subsequences and portions thereof in the host cell. A vector is used herein synonymously with a plasmid and may also include an expression control element for expression in a host cell (e.g., expression vector). Plasmids and vectors generally contain at least an origin of replication for propagation in a cell and a promoter. Plasmids and vectors are therefore useful for genetic manipulation and expression of Dengue virus (DV) peptides, subsequences and portions thereof. Accordingly, vectors that include nucleic acids encoding or complementary to Dengue virus (DV) peptides, subsequences and portions thereof, are provided.

In accordance with the invention, there are provided host cells that express or are transformed with a nucleic acid that encode Dengue virus (DV) peptides, subsequences and portions thereof. Host cells include but are not limited to prokaryotic and eukaryotic cells such as bacteria, fungi (yeast), plant, insect, and animal (e.g., mammalian, including primate and human, CHO cells and hybridomas) cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression. The cells may be a primary cell isolate, cell culture (e.g., passaged, established or immortalized cell line), or part of a plurality of cells, or a tissue or organ ex vivo or in a subject (in vivo).

The term “transformed” or “transfected” when used in reference to a cell (e g., a host cell) or organism, means a genetic change in a cell following incorporation of an exogenous molecule, for example, a protein or nucleic acid (e.g., a transgene) into the cell. Thus, a “transfected” or “transformed” cell is a cell into which, or a progeny thereof in which an exogenous molecule has been introduced by the hand of man, for example, by recombinant DNA techniques.

The nucleic acid or protein can be stably or transiently transfected or transformed (expressed) in the host cell and progeny thereof. The cell(s) can be propagated and the introduced protein expressed, or nucleic acid transcribed. A progeny of a transfected or transformed cell may not be identical to the parent cell, since there may be mutations that occur during replication.

Introduction of Dengue virus (DV) peptides, subsequences and portions thereof, and nucleic acid into target cells (e.g., host cells) can also be carried out by methods known in the art such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinyl acetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.” Accordingly, viral and non-viral vector means delivery into cells, tissue or organs, in vitro, in vivo and ex vivo are included.

In accordance with the invention, treatment methods are provided that include therapeutic (following Dengue virus (DV) infection) and prophylactic (prior to Dengue virus (DV) infection or pathology) methods. For example, methods of treating a subject with a Dengue virus (DV) infection, and methods of protecting a subject from a Dengue virus (DV) infection (e.g., provide the subject with protection against Dengue virus (DV) infection), to decrease or reduce the probability of a Dengue virus (DV) infection in a subject, to decrease or reduce susceptibility of a subject to a Dengue virus (DV) infection, or to inhibit or prevent a Dengue virus (DV) infection in a subject, and to decrease, reduce, inhibit or suppress transmission of the Dengue virus (DV) from a host (e.g., a mosquito) to a subject.

In one embodiment, a method includes administering to a subject an amount of Dengue virus (DV) peptide, subsequence or portion thereof sufficient to treat the subject for the Dengue virus (DV) infection or pathology. In another embodiment, a method includes administering to a subject an amount of a Dengue virus (DV) T cell epitope sufficient to provide the subject with protection against the Dengue virus (DV) infection or pathology, or one or more physiological conditions, disorders, illness, diseases or symptoms caused by or associated with the virus infection or pathology. In a further embodiment, a method includes administering a subject an amount of a Dengue virus (DV) T cell epitope sufficient to treat the subject for the Dengue virus (DV) infection.

Therapeutic and prophylactic methods of treating a subject for a Dengue virus (DV) infection include, for example, treatment of a subject having or at risk of having a Dengue virus (DV) infection or pathology. Such methods include administering Dengue virus (DV) peptide, subsequence or portion thereof to therapeutically or prophylactically (vaccinating or immunizing) treat a subject having or at risk of having a Dengue virus (DV) infection or pathology. Such methods can treat the Dengue virus (DV) infection or pathology, or provide the subject with protection from infection (e.g., prophylactic protection). Dengue virus (DV) peptides, subsequences and portions thereof include T cell epitopes. In one embodiment, a method includes administering an amount of Dengue virus (DV) peptide, subsequence or portion thereof (e.g., a T cell epitope) to a subject in need thereof, sufficient to provide the subject with protection against Dengue virus (DV) infection or pathology. In another embodiment, a method includes administering an amount of a Dengue virus (DV) peptide, subsequence or portion thereof (e.g., a T cell epitope) to a subject in need thereof sufficient to treat, vaccinate or immunize the subject against the Dengue virus (DV) infection or pathology.

In methods of the invention, any Dengue virus (DV) peptide, subsequence or portion thereof can be administered. Non-limiting examples include Dengue virus (DV) peptide, subsequence or portion thereof of a DENV1, DENV2, DENV3 or DENV4 serotype. Additional non-limiting examples include a Dengue virus structural protein (e.g., C, M or E) or non-structural (NS) protein (e.g., NS1, NS2A, NS2B, NS3, NS4A, NS4B or NS5) T cell epitope. Further non-limiting examples include a peptide sequence set forth as: GMLQGRGPL (SEQ ID NO:1); VAFLRFLTI (SEQ ID NO:2); RALIFILL (SEQ ID NO:3); MTMRCIGI (SEQ ID NO:4); VSWTMKIL (SEQ ID NO:5); RLITVNPIV (SEQ ID NO:13); FSLGVLGM (SEQ ID NO:6); VAVSFVTLI (SEQ ID NO:7); LAVTIMAIL (SEQ ID NO:8); TAIANQATV (SEQ ID NO:9); TAIANQATV (SEQ ID NO:10); YSQVNPITL (SEQ ID NO:11); RMLINRFTM (SEQ ID NO:12); or KLAEAIFKL (SEQ ID NO:14), a subsequence thereof or an amino acid substitution thereof.

In particular methods embodiments, one or more disorders, diseases, physiological conditions, pathologies and symptoms associated with or caused by a Dengue virus (DV) infection or pathology will respond to treatment. In particular methods embodiments, treatment methods reduce, decrease, suppress, limit, control or inhibit Dengue virus (DV) numbers or titer; reduce, decrease, suppress, limit, control or inhibit pathogen proliferation or replication; reduce, decrease, suppress, limit, control or inhibit the amount of a pathogen protein; or reduce, decrease, suppress, limit, control or inhibit the amount of a Dengue virus (DV) nucleic acid. In additional particular methods embodiments, treatment methods include an amount of a Dengue virus (DV) peptide, subsequence or portion thereof sufficient to increase, induce, enhance, augment, promote or stimulate an immune response against a Dengue virus (DV); increase, induce, enhance, augment, promote or stimulate Dengue virus (DV) clearance or removal; or decrease, reduce, inhibit, suppress, prevent, control, or limit transmission of Dengue virus (DV) to a subject (e.g., transmission from a host, such as a mosquito, to a subject). In further particular methods embodiments, treatment methods include an amount of Dengue virus (DV) peptide, subsequence or portion thereof sufficient to protect a subject from a Dengue virus (DV) infection or pathology, or reduce, decrease, limit, control or inhibit susceptibility to Dengue virus (DV) infection or pathology.

Methods of the invention include treatment methods, which result in any therapeutic or beneficial effect. In various methods embodiments, Dengue virus (DV) infection, proliferation or pathogenesis is reduced, decreased, inhibited, limited, delayed or prevented, or a method decreases, reduces, inhibits, suppresses, prevents, controls or limits one or more adverse (e.g., physical) symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In additional various particular embodiments, treatment methods include reducing, decreasing, inhibiting, delaying or preventing onset, progression, frequency, duration, severity, probability or susceptibility of one or more adverse symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In further various particular embodiments, treatment methods include accelerating, facilitating, enhancing, augmenting, or hastening recovery of a subject from a Dengue virus (DV) infection or pathogenesis, or one or more adverse symptoms, disorders, illnesses, diseases or complications caused by or associated with Dengue virus (DV) infection, proliferation or replication, or pathology (e.g., fever, rash, headache, pain behind the eyes, muscle or joint pain, nausea, vomiting, loss of appetite). In yet additional various embodiments, treatment methods include stabilizing infection, proliferation, replication, pathogenesis, or an adverse symptom, disorder, illness, disease or complication caused by or associated with Dengue virus (DV) infection, proliferation or replication, or pathology, or decreasing, reducing, inhibiting, suppressing, limiting or controlling transmission of Dengue virus (DV) from a host (e.g., mosquito) to an uninfected subject.

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with Dengue virus (DV) infection, proliferation or replication, or pathology (e.g., fever, rash, headache, pain behind the eyes, muscle or joint pain, nausea, vomiting, loss of appetite). Thus, a satisfactory clinical endpoint is achieved when there is an incremental improvement or a partial reduction in an adverse symptom, disorder, illness, disease or complication caused by or associated with Dengue virus (DV) infection, proliferation or replication, or pathology, or an inhibition, decrease, reduction, suppression, prevention, limit or control of worsening or progression of one or more adverse symptoms, disorders, illnesses, diseases or complications caused by or associated with Dengue virus (DV) infection, Dengue virus (DV) numbers, titers, proliferation or replication, Dengue virus (DV) protein or nucleic acid, or Dengue virus (DV) pathology, over a short or long duration (hours, days, weeks, months, etc.).

A therapeutic or beneficial effect also includes reducing or eliminating the need, dosage frequency or amount of a second active such as another drug or other agent (e.g., small molecule, protein) used for treating a subject having or at risk of having a Dengue virus (DV) infection or pathology. For example, reducing an amount of an adjunct therapy, for example, a reduction or decrease of a treatment for a Dengue virus (DV) infection or pathology, or a vaccination or immunization protocol is considered a beneficial effect. In addition, reducing or decreasing an amount of a Dengue virus (DV) antigen used for vaccination or immunization of a subject to provide protection to the subject is considered a beneficial effect.

Adverse symptoms and complications associated with Dengue virus (DV) infection and pathology include, for example, e.g., fever, rash, headache, pain behind the eyes, muscle or joint pain, nausea, vomiting, loss of appetite, etc. Other symptoms of Dengue virus (DV) infection or pathogenesis are known in the art and treatment thereof in accordance with the invention is provided. Thus, the aforementioned symptoms and complications are treatable in accordance with the invention.

Methods and compositions of the invention also include increasing, stimulating, promoting, enhancing, augmenting or inducing an anti-pathogen CD8⁺ or CD4⁺ T cell response in a subject with or at risk of a Dengue virus infection or pathology. In one embodiment, a method includes administering to a subject an amount of Dengue virus (DV) peptide, subsequence or portion thereof sufficient to increase, stimulate, promote, enhance, augment or induce anti-pathogen CD8⁺ or CD4⁺ T cell response in the subject. In another embodiment, a method includes administering to a subject an amount of Dengue virus (DV) peptide, subsequence or portion thereof and administering a Dengue virus (DV) antigen, live or attenuated Dengue virus (DV), or a nucleic acid encoding all or a portion (e.g., a T cell epitope) of any protein or proteinaceous Dengue virus (DV) antigen sufficient to increase, stimulate, promote, enhance, augment or induce anti-Dengue virus (DV) CD8⁺ or CD4⁺ T cell response in the subject.

Methods of the invention additionally include, among other things, increasing production of a Th1 cytokine (e.g., interferon gamma, IL-2, TNF-alpha, etc.). In one embodiment, a method includes administering to a subject in need thereof an amount of Dengue virus (DV) peptide, subsequence or portion thereof sufficient to increase production of a Th1 cytokine in the subject (e.g., interferon gamma, IL-2, TNF-alpha, etc.).

Methods and compositions of the invention include administration of Dengue virus (DV) peptide, subsequence or portion thereof to a subject prior to contact, exposure or infection by a Dengue virus, administration prior to, substantially contemporaneously with or after a subject has been contacted by, exposed to or infected with a Dengue virus (DV), and administration prior to, substantially contemporaneously with or after Dengue virus (DV) pathology or development of one or more adverse symptoms. Methods and compositions of the invention also include administration of Dengue virus (DV) peptide, subsequence or portion thereof to a subject prior to, substantially contemporaneously with or following a Dengue virus (DV) peptide, subsequence or portion thereof or adverse symptom, disorder, illness or disease caused by or associated with a Dengue virus (DV) infection, or pathology. A subject infected with a Dengue virus (DV) may have an infection over a period of days, months, or years.

Invention compositions (e.g., Dengue virus (DV) peptide, subsequence or portion thereof, including T cell epitopes) and methods can be combined with any compound, agent, drug, treatment or other therapeutic regimen or protocol having a desired therapeutic, beneficial, additive, synergistic or complementary activity or effect. Exemplary combination compositions and treatments include second actives, such as anti-Dengue virus (DV) compounds, agents and drugs, as well as agents that assist, promote, stimulate or enhance efficacy. Such anti-Dengue virus (DV) drugs, agents, treatments and therapies can be administered or performed prior to, substantially contemporaneously with or following any other method of the invention, for example, a therapeutic method of treating a subject for a Dengue virus (DV) infection or pathology, or a method of prophylactic treatment of a subject for a Dengue virus (DV) infection.

Dengue virus (DV) peptides, subsequences and portions thereof can be administered as a combination composition, or administered separately, such as concurrently or in series or sequentially (prior to or following) administering a second active, to a subject. The invention therefore provides combinations in which a method of the invention is used in a combination with any compound, agent, drug, therapeutic regimen, treatment protocol, process, remedy or composition, such as an anti-Dengue virus (DV) or immune stimulating, enhancing or augmenting protocol, or pathogen vaccination or immunization (e.g., prophylaxis) set forth herein or known in the art. The compound, agent, drug, therapeutic regimen, treatment protocol, process, remedy or composition can be administered or performed prior to, substantially contemporaneously with or following administration of Dengue virus (DV) peptide, subsequence or portion thereof, or a nucleic acid encoding all or a portion (e.g., a T cell epitope) of a Dengue virus (DV) peptide, subsequence or portion thereof, to a subject. Specific non-limiting examples of combination embodiments therefore include the foregoing or other compound, agent, drug, therapeutic regimen, treatment protocol, process, remedy or composition.

An exemplary combination is a Dengue virus (DV) peptide, subsequence or portion thereof (e.g., a T cell epitope) and a different Dengue virus (DV) peptide, subsequence or portion thereof (e.g., a different T cell epitope), antigen (e.g., Dengue virus (DV) extract), or live or attenuated Dengue virus (DV) (e.g., inactivated Dengue virus (DV)). Such Dengue virus (DV) antigens set forth herein or known to one of skill in the art include a Dengue virus (DV) antigen that increases, stimulates, enhances, promotes, augments or induces a proinflammatory or adaptive immune response, numbers or activation of an immune cell (e.g., T cell, natural killer T (NKT) cell, dendritic cell (DC), B cell, macrophage, neutrophil, eosinophil, mast cell, CD4⁺ or a CD8⁺ cell, B220⁺ cell, CD14⁺, CD11b⁺ or CD11c⁺ cells), an anti-Dengue virus (DV) CD4⁺ or CD8⁺ T cell response, production of a Th1 cytokine, a T cell mediated immune response, etc.

Combination methods embodiments include, for example, second actives such as anti-pathogen drugs, such as protease inhibitors, reverse transcriptase inhibitors, virus fusion inhibitors and virus entry inhibitors, antibodies to pathogen proteins, live or attenuated pathogen, or a nucleic acid encoding all or a portion (e.g., an epitope) of any protein or proteinaceous pathogen antigen, immune stimulating agents, etc., and include contact with, administration in vitro or in vivo, with another compound, agent, treatment or therapeutic regimen appropriate for pathogen infection, vaccination or immunization

Methods of the invention also include, among other things, methods that result in a reduced need or use of another compound, agent, drug, therapeutic regimen, treatment protocol, process, or remedy. For example, for a Dengue virus (DV) infection or pathology, vaccination or immunization, a method of the invention has a therapeutic benefit if in a given subject a less frequent or reduced dose or elimination of an anti-Dengue virus (DV) treatment results. Thus, in accordance with the invention, methods of reducing need or use of a treatment or therapy for a Dengue virus (DV) infection or pathology, or vaccination or immunization, are provided.

In invention methods in which there is a desired outcome, such as a therapeutic or prophylactic method that provides a benefit from treatment, vaccination or immunization Dengue virus (DV) peptide, subsequence or portion thereof can be administered in a sufficient or effective amount. As used herein, a “sufficient amount” or “effective amount” or an “amount sufficient” or an “amount effective” refers to an amount that provides, in single or multiple doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by Dengue virus (DV) peptide, subsequence or portion thereof alone, in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

The term “subject” refers to an animal, typically a mammalian animal (mammal), such as a non human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans. Subjects include animal disease models, for example, mouse and other animal models of pathogen infection and reactivation from latency known in the art.

Subjects appropriate for treatment include those having or at risk of having Dengue virus infection or pathology. Target subjects therefore include subjects that have been exposed to or contacted with Dengue virus (DV), or that have an ongoing infection or have developed one or more adverse symptoms caused by or associated with Dengue virus (DV) infection or pathology, regardless of the type, timing or degree of onset, progression, severity, frequency, duration of the symptoms.

Target subjects also include those at risk of Dengue virus (DV) exposure, contact, infection or pathology or at risk of having or developing a Dengue virus (DV) infection or pathology. The invention methods are therefore applicable to treating a subject who is at risk of Dengue virus (DV) exposure, contact, infection or pathology, but has not yet been exposed to or contacted with Dengue virus (DV). Prophylactic methods are therefore included. Target subjects for prophylaxis can be at increased risk (probability or susceptibility) of exposure, contact, infection or pathology, as set forth herein and known in the art. Such subjects are considered in need of treatment due to being at risk.

Target subjects for prophylaxis need not be at increased risk but may be from the general population in which it is desired to vaccinate or immunize a subject against a Dengue virus (DV) infection, for example, an child such as an infant or toddler in which it is desired to vaccinate or immunize against a Dengue virus (DV) can be administered Dengue virus (DV) peptide, subsequence or portion thereof. In another non-limiting example, a subject that is not specifically at risk of exposure to or contact with a Dengue virus (DV), but nevertheless desires protect against infection or pathology, can be administered a Dengue virus (DV) peptide, subsequence or portion thereof. Such subjects are also considered in need of treatment.

At risk subjects appropriate for treatment also include subjects exposed to environments in which subjects are at risk of a Dengue virus (DV) infection due to a mosquito bite. Subjects appropriate for treatment therefore include human subjects exposed to mosquitos, or travelling to regions or countries in which Dengue virus (DV) is know to infect subjects due, for example, transmission from mosquitos present in thois regiosn or countries. At risk subjects appropriate for treatment also include subjects where the risk of Dengue virus (DV) infection or pathology is increased due to changes in infectivity or the type of region of Dengue virus (DV) carrying mosquitos. Such subjects are also considered in need of treatment due to such a risk.

“Prophylaxis” and grammatical variations thereof mean a method in which contact, administration or in vivo delivery to a subject is prior to contact with or exposure to or infection. In certain situations it may not be known that a subject has been contacted with or exposed to Dengue virus (DV), but administration or in vivo delivery to a subject can be performed prior to infection or manifestation of pathology (or an associated adverse symptom, condition, complication, etc. caused by or associated with a Dengue virus (DV)). For example, a subject can be immunized or vaccinated with a Dengue virus (DV) peptide, subsequence or portion thereof. In such case, a method can eliminate, prevent, inhibit, suppress, limit, decrease or reduce the probability of or susceptibility towards a Dengue virus (DV) infection or pathology, or an adverse symptom, condition or complication associated with or caused by or associated with a Dengue virus (DV) infection or pathology.

Treatment of an infection can be at any time during the infection. Methods of the invention may be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intrapleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, intralymphatic.

Dengue virus (DV) peptide, subsequence or portion thereof can be administered as a combination (e.g., with a second active), or separately concurrently or in sequence (sequentially) in accordance with the methods as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by Dengue virus (DV) infection, pathology, or an adverse symptom, condition or complication associated with or caused by a Dengue virus (DV). Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

Doses can be based upon current existing protocols, empirically determined, using animal disease models or optionally in human clinical trials. Initial study doses can be based upon animal studies set forth herein, for a mouse, which weighs about 30 grams, and the amount of Dengue virus (DV) peptide, subsequence or portion thereof administered that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg,100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, the onset, progression, severity, frequency, duration probability of or susceptibility of the symptom, condition, pathology or complication the type of pathogen infection or pathogenesis, reactivation from latency or vaccination or immunization to which treatment is directed, the clinical endpoint desired, previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

Typically, for therapeutic treatment, Dengue virus (DV) peptide, subsequence or portion thereof will be administered as soon as practical, typically within 1-2, 2-4, 4-12, 12-24 or 24-72 hours after a subject is exposed to or contacted with a Dengue virus (DV), or within 1-2, 2-4, 4-12, 12-24 or 24-48 hours after onset or development of one or more adverse symptoms, conditions, pathologies, complications, etc., associated with or caused by a Dengue virus (DV) infection or pathology. For prophylactic treatment in connection with vaccination or immunization, Dengue virus (DV) peptide, subsequence or portion thereof can be administered for a duration of 0-4 weeks, e.g., 2-3 weeks, prior to exposure to, contact or infection with Dengue virus (DV), or at least within 1-2, 2-4, 4-12, 12-24, 24-48 or 48-72 hours prior to exposure to, contact or infection with Dengue virus (DV). For an acute infection, Dengue virus (DV) peptide, subsequence or portion thereof is administered at any appropriate time.

The dose amount, number, frequency or duration may be proportionally increased or reduced, as indicated by the status of the subject. For example, whether the subject has a pathogen infection, whether the subject has been exposed to, contacted or infected with pathogen or is merely at risk of pathogen contact, exposure or infection, whether the subject is or is at risk of suffering from reactivation from latency or whether the subject is a candidate for or will be vaccinated or immunized. The dose amount, number, frequency or duration may be proportionally increased or reduced, as indicated by any adverse side effects, complications or other risk factors of the treatment or therapy.

Dengue virus (DV) peptides, subsequences and portions thereof can be incorporated into pharmaceutical compositions, e.g., a pharmaceutically acceptable carrier or excipient. Such pharmaceutical compositions are useful for, among other things, administration to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Carriers which may be used include Vaseline, lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

Dengue virus (DV) peptides, subsequences and portions thereof, along with any adjunct agent, compound drug, composition, whether active or inactive, etc., can be packaged in unit dosage form (capsules, tablets, troches, cachets, lozenges) for ease of administration and uniformity of dosage. A “unit dosage form” as used herein refers to physically discrete units suited as unitary dosages for the subject to be treated; each unit containing a predetermined quantity of active ingredient optionally in association with a pharmaceutical carrier (excipient, diluent, vehicle or filling agent) which, when administered in one or more doses, is calculated to produce a desired effect (e.g., prophylactic or therapeutic effect). Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

The invention provides kits that include Dengue virus (DV) peptide, subsequence or portion thereof, optionally with a second active, and pharmaceutical formulations thereof, packaged into suitable packaging material. A kit typically includes a label or packaging insert including a description of the components or instructions for use in vitro, in vivo, or ex vivo, of the components therein. A kit can contain a collection of such components, e.g., Dengue virus (DV) peptide, subsequence or portion thereof and optionally a second active, such as another compound, agent, drug or composition.

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a disk (e.g., hard disk, flash memory), optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder or disease (e.g., viral infection, vaccination or immunization) for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or prophylactic or therapeutic regimes described herein. Exemplary instructions include, instructions for treating a Dengue virus (DV) infection or pathology, and instructions for providing a subject with protection against Dengue virus (DV) infection or pathology.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise. Thus, for example, reference to an “Dengue virus (DV) peptide, subsequence or portion thereof” or a “Dengue virus (DV)” includes a plurality of Dengue virus (DV) peptides, subsequences and portions thereof or serotypes of Dengue virus (DV), and reference to an “activity or function” can include reference to one or more activities or functions of a Dengue virus (DV) peptide, subsequence or portion thereof including function as a T cell epitopes, an ability to elicit a measurable or detectable anti-DV CD8⁺ T cell response, and so forth.

As used herein, all numerical values or ranges include fractions of the values and integers within such ranges and fractions of the integers within such ranges unless the context clearly indicates otherwise. Thus, to illustrate, reference to a numerical range, such as a percentage range, 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

Reference to a series of ranges include ranges which combine the values of the boundaries of different ranges within the series. Thus, to illustrate reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, procedures, assays or analysis. For example, in certain embodiments or aspects of the invention, antibodies or other materials and method steps are excluded. In certain embodiments and aspects of the invention, for example, a Dengue virus (DV) peptide, subsequence or portion thereof is excluded. Thus, even though the invention is generally not expressed herein in terms of what is not included, embodiments and aspects that expressly exclude compositions (e.g., antibodies or pathogen antigens) or method steps are nevertheless disclosed and included in the invention.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

### This Example Includes a Description of Materials and Methods.

### Mice and Infections

C57BL/6 (H-2^{b}) mice were from The Jackson Laboratory and were subsequently bred at the animal facility at La Jolla Institute for Allergy and Immunology (LIAI). IFN-α/βR^{-/-} mice on the C57BL/6 background were obtained from Wayne Yokoyama (Washington University, St. Louis, Mo.) via Carl Ware (LIAI). B6.SJL mice were purchased from The Jackson Laboratory or Taconic. Mice were used between 5 and 8 weeks of age. Mice were infected i.v. in the lateral tail vein with 200 μl S221 in 5% FBS/PBS. Blood was obtained from anesthetized mice by retro-orbital puncture.

### Cell Culture and Viral Stocks

The hybridoma clones SFR3, GK1.5 and 2.43, which produce rat anti-human HLA DR5, anti-mouse CD4 and anti-mouse CD8 IgG2b Ab, respectively, were from the American Type Culture Collection (ATCC). The hybridoma cell lines were grown in protein-free hybridoma medium supplemented with penicillin, streptomycin, HEPES, GlutaMAX, and 2-ME (all from Invitrogen) at 37° C. 5% CO₂. C6/36, an Aedes albopictus cell line, was cultured in Leibovitz's L-15 Medium (Invitrogen) supplemented with 10% fetal bovine serum (FBS) (Gemini Bio-Products), penicillin, streptomycin, and HEPES at 28° C. in the absence of CO₂. DENV2 strain PL046 is Taiwanese clinical isolate and was obtained from Dr. Huan-Yao Lei (National Cheng Kung University, Taiwan) (Lin et al., J Virol 72:9729 (1998)). Alternate passaging of PL046 through the sera of IFN-α/βR^{-/-}×IFN-γR^{-/-} mice and C6/36 cells resulted in a novel DENV2 strain, termed D2S10, that causes more severe disease in those mice (Shresta et al., J Virol 80:10208 (2006)). S221 is a triple-plaque-purified clone from the D2S10 population which differs from PL046 at amino acid positions E₁₂₄, E₁₂₈, and NS1₂₇₈.

Viral stocks were obtained by amplification in C6/36 cells as described previously (Diamond et al., J Virol 74:7814 (2000)). Briefly, C6/36 monolayers were infected with DENV2 and cultured for 7 days. Virus in the supernatant was concentrated by ultracentrifugation at 50,000 g for 90 min, resuspended in 20% FBS/PBS, and stored at −80° C. Infectious doses were determined based on genomic equivalents (GE) which were quantified by real-time RT-PCR. There are approximately 10³ GE per PFU for PL046 and 10⁴ GE/PFU for S221 (PFU determined by plaque assay on baby hamster kidney cells).

### Bioinformatic Analyses

Data of peptides binding to H-2^{b} molecules were used to develop binding predictions. The predictions were performed, and the data set available at the time comprised 521 eight-mer (8-mer) peptides binding to K^{b} and 319 nine-mer (9-mer) peptides binding to D^{b}. In addition, combinatorial peptide libraries described in reference (Udaka et al., Immunogenetics 51:816 (2000)) were available for 8-mer peptides binding to K^{b} and 9-mer peptides binding to D^{b}. These two sources of data were combined to calculate scoring matrices that quantify the contribution of each residue in a fixed-length peptide to binding to an MHC molecule, as described previously (Peters et al., BMC Bioinformatics 6:132 (2005)). The entire DENV2 polyprotein PL046 was then scanned using these matrices for peptides binding to either MHC molecule with a predicted affinity of IC₅₀ <500 nM. This approach selected 55 8-mer peptides predicted to bind H-2 K^{b} and 51 nine-mer peptides predicted to bind H-2 D^{b}.

### Peptide Synthesis

Peptides were synthesized as crude material by Pepscan Systems (Lelystad, the Netherlands) as described (Sidney et al., Immunome Res 4:2 (2008)). One-hundred and six 8- and 9-mer peptides were made and combined into 10 pools. Peptides used in the binding assays and immunizations studies were synthesized (A and A Labs, San Diego, Calif.) and purified to ≧95% homogeneity by reverse-phase high-performance liquid chromatography (HPLC). Peptide purity was determined using analytical reverse phase-HPLC and amino acid analysis, sequencing, and/or mass spectrometry. Peptides were radiolabeled with the chloramine T method, as described (Sidney et al., Curr Protoc Immunol 18:1813 2001)).

### Major Histocompatiblity Complex (MHC) Peptide-Binding Assays

MHC purification and quantitative assays to measure the binding affinity of peptides to purified H-2 K^{b} and H-2 D^{b} molecules were performed as previously described (Sidney et al., Curr Protoc Immunol 18:1813 2001); Vitiello et al., J Immunol 157:5555 (1996)). Briefly, 0.1-1 nM of radiolabeled peptide was co-incubated at room temperature with 1 μM to 1 nM of purified MHC in the presence of 1-3 μM human β₂-microglobulin (Scripps Laboratories) and a mixture of protease inhibitors. After a 2 day incubation, binding of the radiolabeled peptide to the corresponding MHC class I molecule was determined by capturing MHC/peptide complexes on Lumitrac 600 microplates (Greiner Bio-One) coated with either the Y3 (anti-H-2 K^{b}) or 28-14-8S (anti-H-2 D^{b}, L^{d} and D^{q}) Ab, followed by measurement of bound counts per minute (cpm) using the TopCount microscintillation counter (Packard).

### IFN-γ Enzyme Linked Immunosorbent Spot (ELISPOT) Assay

CD8⁺ T cells were isolated by magnetic bead positive selection (Miltenyi) from the spleens of C57BL/6 mice 7 or 8 days after infection with 10¹¹ GE PL046 or S221. CD8⁺ T cells (10⁵) were stimulated with 7.5×10⁵ uninfected splenocytes as antigen-presenting cells (APCs) and 1 μg of individual DENV2 peptides in 96-well flat-bottom plates (Immobilon-P, Millipore) coated with anti-IFN-γ monoclonal antibody (mAb) (Mabtech). Following 20 hour incubation at 37° C. the wells were washed with PBS/0.05% Tween and then incubated with biotinylated IFN-γ mAb (Mabtech) for 3 hour. The spots were developed using Vectastain ABC peroxidase (Vector Laboratories) and 3-amino-9-ethyl carbazol (AEC) (Sigma-Aldrich) and counted by computer-assisted image analysis (Zeiss KS ELISPOT reader). The response against an irrelevant, vaccinia virus-derived control peptide (VACV-WR B6R 108-116, K^{b}-restricted) was subtracted from the responses to each individual DENV2 peptide to obtain the net number of spots. The criteria for positivity were a stimulation index of 2.0, net SFC/10⁶ cells of ≧20, and p<0.05 when compared with the irrelevant peptide.

### Flow Cytometric Analyses

All Ab were purchased from eBioscience with the exception of anti-CD8α-PerCP and anti-CD107a-FITC which were obtained from BD Biosciences. RBC in blood were lysed using RBC lysis buffer (eBioscience). For surface staining, splenocytes or blood cells were washed, incubated with supernatant from 2.4G2-producing hybridoma cells to block Fc receptors, and labeled with anti-CD8α-PerCP, anti-CD44-APC, and anti-CD62L-PE. For intracellular cytokine staining, 10⁶ splenocytes or 10⁵ blood cells were plated in 96-well U-bottom plates and stimulated with individual DENV peptides (0.1 μg/ml) in the presence of brefeldin A (GolgiPlug, BD Biosciences) for 5 h. For CD107a staining, CD107a-FITC (or rat IgG2a-FITC) was added to the wells at the same time as the peptide. Cells were washed, incubated with 2.4G2 supernatant, labeled with anti-CD8α-PerCP, fixed and permeabilized using the BD Cytofix/Cytoperm Kit, and stained with anti-IFN-γ-APC and anti-TNF-α-PE. Samples were read on a FACS Caliber (BD Biosciences) and analyzed using FloJo software (Tree Star, Inc.)

### CD4⁺ and CD8⁺ T Cell Depletions

Hybridoma supernatants were clarified by centrifugation, concentrated with Amicon filters (Millipore), and protein G-purified (Pierce). Antibody preparations were quantified by spectrophotometry. IFN-α/βR^{-/-} mice were given an intraperitoneal (i.p.) injection of 250 μg of SFR3, GK1.5 or 2.43 in phosphate buffered saline (PBS 250 μl total volume) 3 days and 1 day before infection. At day 6 after infection 85-95% of CD4⁺ and/or CD8⁺ cells had been depleted.

### Peptide Immunization

FN-α/βR^{-/-} mice were immunized subcutaneously (s.c.) at the base of the tail with 50 μg of each DENV2 peptide plus 100 μg helper peptide (IA^{b}-restricted hepatitis B virus core 128-140) emulsified in incomplete Freund's adjuvant (IFA) (Difco). Mock-immunized mice received helper peptide and DMSO emulsified in IFA. The final concentration of DMSO was 13%. Mice were infected 12 days after immunization.

In vivo Cytotoxicity Assay

IFN-a/bR^{-/-} mice (recipients) were infected with 10¹⁰ GE S221. Splenocytes and lymph node cells (targets) were harvested from donor B6.SJL congenic mice (CD45.1) 7 days later. Red blood cells (RBC) were lysed, and the target cells were pulsed with 0.5 μg/ml of the irrelevant vaccinia virus (VACV) peptide, C₅₁₋₅₉, NS2A₈₋₁₅, NS4B₉₉₋₁₀₇, NS5₂₃₇₋₄₅ or a pool of C₅₁₋₅₉, NS2A₈₋₁₅, NS⁴B₉₉₋₁₀₇, and NS5₇₃₇₋₄₅ for 1 h at 37° C. The cells were then washed and labeled with CFSE (Invitrogen) in PBS/0.1% BSA for 10 min at 37° C. DENV peptide-pulsed cells were labeled with 1 μM CFSE (CFSE^{high}) and the irrelevant peptide-pulsed cells with 100 nM CFSE (CFSE^{low}). After washing, the two cell populations were mixed at a 1:1 ratio and 5×10⁶ cells from each population were injected intravenously (i.v.) into naïve or infected recipient mice. After 4 hour, the mice were sacrificed and splenocytes stained with CD45.1-allophycocyanin and analyzed by flow cytometry, gating on CD45.1 cells. Percentage killing was calculated as follows: 100−((percentage DENV peptide-pulsed in infected mice/percentage irrelevant peptide-pulsed in infected mice)/(percentage DENV peptide-pulsed in naïve mice/percentage irrelevant peptide-pulsed in naïve mice)×100.

### Quantitation of DENV Burden in Mice

Mice were euthanized by isoflurane inhalation and blood was collected via cardiac puncture. Serum was separated from whole blood by centrifugation in serum separator tubes (Starsted). Small tissue pieces were immediately placed into RNAlater (Qiagen) and subsequently homogenized for 3 min at 4° C. in 1 ml tissue lysis buffer (Qiagen Buffer RLT) using a Mini-Beadbeater-8 (BioSpec Products). Immediately after homogenization, all tissues were centrifuged (5 min, 4° C., 16,000×g) to pellet debris, and RNA was isolated using the RNeasy Mini Kit (Qiagen). Serum RNA was isolated using the QIAamp Viral RNA Mini Kit (Qiagen). After elution, viral RNA was snap-frozen in liquid N₂ and stored at −80° C. until measured by real-time RT-PCR.

### Quantitative Real-Time RT-PCR

Quantitative RT:PCR was performed according to a published protocol (Houng et al., J Virol Methods 86:1 (2000)), except a MyiQ Single-Color Real-Time PCR Detection System (Bio-Rad) with iScript One-Step RT-PCR kit for Probes, (Bio-Rad) were used. The DENV standard curve was generated with serial dilutions of a known concentration of DENV genomic RNA which was in vitro transcribed (MAXIscript Kit, Ambion) from a plasmid containing the cDNA template of PL046 3′UTR. After transcription, DNA was digested with DNase I, and RNA was purified using the RNeasy Mini Kit (Qiagen) and quantified by UV spectrophotometry. To control for RNA quality and quantity when measuring DENV2 in tissues, the level of 18S rRNA was measured using an 18S rRNA Control Kit (Eurogentec) in parallel real-time RT-PCR reactions. A relative 18S standard curve was made from total splenic RNA.

### Statistical Analyses

Data were analyzed with Prism software version 5.0 (GraphPad Software, Inc.). Statistical significance for CD8⁺ T cell numbers and viral load were determined using the unpaired t-test with Welch's correction; for the ELISPOT, the unpaired t-test was used.

### Example 2

### This Example Includes a Description of the Identification of DENV2-Derived Epitopes Recognized by CD8⁺ T Cells.

In order to map the specificity of the CD8⁺ T cell response to dengue virus in C57BL/6 mice, the proteome of the DENV2 clinical isolate, PL046, which is approximately 3390 amino acids and encodes three structural (core (C), envelope (E), and membrane (M)), and seven non-structural (NS) (NS1, NS2A, NS2B, NS3, NS4A, NS4B, NS5) proteins, was screened for the presence of peptides predicted to bind H-2^{b} class I molecules (K^{b} and D^{b}) with high affinity using a bioinformatics prediction method (Kotturi et al., J Virol 81:4928 (2007)). 106 possible MHC class I (H-2^{b}) binding peptides were identified. These peptides were combined into 10 pools and tested in IFN-γ ELISPOT assays using CD8⁺ T cells from PL046-infected C57BL/6 mice or S221-infected C57BL/r mice. Positive pools were deconvoluted and individual peptides tested by IFN-γ ELISPOT. The IFN-γ ELISPOT testing was performed using CD8⁺ T cells isolated from C57BL/6 mice seven days after infection with 10¹¹ genomic equivalents (GE) of the DENV2 strains PL046 (clinical isolate) or S221 (mouse serum-passaged).

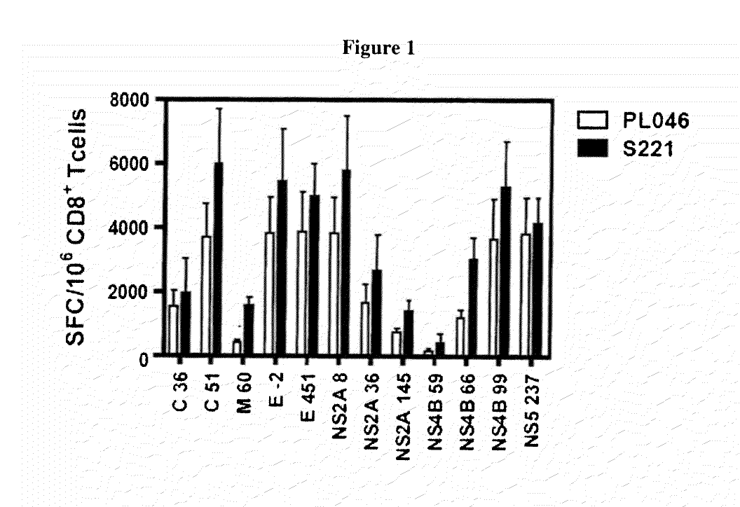

Referring to FIG. 1, the data are expressed as the mean number of net spot-forming cells (SFC) per 10⁶ CD8⁺ T cells. Three independent studies performed in triplicate were averaged and the error bars represent the SEM. The criteria for positivity were a stimulation index of ≧2.0, p<0.05 when compared with an irrelevant control peptide, and net SFC/10⁶ cells of ≧20. The 12 peptides shown are positive for both virus strains (FIG. 1).

The 12 positive peptides identified include epitopes derived from 6 of the 10 DENV proteins, including all three structural DENV proteins (C, M, E) and three non-structural proteins (NS2A, NS4B, and NS5). The highest responses were observed against C₅₁₋₅₉, E₂₋₆, E₄₅₁₋₄₅₈, NS2A₈₋₁₅, NS4B₉₉₋₁₀₇, and NS⁵₂₃₇₋₂₄₅. Major, intermediate, and minor CD8⁺ T cell epitopes were identified. No peptides were identified from the remaining proteins, although numerous predicted candidate epitopes were predicted and studied.

S221 is a biological clone of D2S10, a novel DENV2 strain that was obtained by alternately passaging PL046 between the sera of mice and mosquito cells, and was found to be more pathogenic in doubly-deficient IFN-α/βR^{-/-}×IFN-γR^{-/-} mice than PL046 (Shresta et al., J Virol 80:10208 (2006)). S221 differs from PL046 at residues E 124, E 128, and NS1 228, which are not residues found in the 106 predicted epitopes. The 12 positive peptides identified for PL046 were also positive for S221 (FIG. 1). Because the response to each DENV2 peptide was higher in S221 infected mice compared with PL046-infected mice, S221 mice were used in all subsequent studies.

To confirm the MHC class I restriction of the 12 identified epitopes, measurements were performed of their MHC binding capacity using purified K^{b} and D^{b} molecules in an in vitro binding assay. The results are shown in Table I (below). Seven of the 12 epitopes bound the predicted allele with either high or intermediate affinity, as indicated by an IC₅₀ of <50 nM or <500 nM, respectively. The five remaining epitopes also bound with biologically relevant affinities in the 500-2000 nM range.

The amino acid sequence of each positive peptide (SEQ ID NOS:1-12) along with its binding affinity to MHC class I, are provided in Table 1. As described above, with exception of five epitopes (E₂₋₆, NS2A₈₋₁₅, C₃₆₋₄₄, NS2A₁₄₅₋₁₅₃ and NS4B₅₉₋₆₆) the peptide are intermediate or high affinity binders. The most dominant epitope, NS4B₉₉, had a very high binding affinity to H2-D^{b} (1.3 nM). The lower binding affinities of epitopes such as E₂₋₆ and NS2A₈₋₁₅ are likely due to the presence of cysteine (C) and methionine (M) residues which become oxidized in vitro, and thus the in vitro data may not be reflective of in vivo binding.

TABLE 1
DENV2-derived CD8⁺ T cell epitopes.

| | | | Binding affinity | |
|---|---|---|---|---|
| | Peptide | | (IC₅₀ nM) | |
| Epitope | sequence | | H2-D^{b} | H2-K^{b} |
| C₃₆₋₄₄ | GMLQGRGPL | D^{b} 9-mer | 820 | 1336 |
| | (SEQ ID NO: 1) | | | |
| C₅₁₋₅₉ | VAFLRFLTI | K^{b} 9-mer | 1379 | 20 |
| | (SEQ ID NO: 2) | | | |
| M₆₀₋₆₇ | RALIFILL | K^{b} 8-mer | -- | 22 |
| | (SEQ ID NO: 3) | | | |
| E₋₂₋₆ | MTMRCIGI | K^{b} 8-mer | -- | 1085 |
| | (SEQ ID NO: 4) | | | |
| E₄₅₁₋₄₅₈ | VSWTMKIL | K^{b} 8-mer | -- | 458 |
| | (SEQ ID NO: 5) | | | |
| NS2A₈₋₁₅ | FSLGVLGM | K^{b} 8-mer | 1803 | -- |
| | (SEQ ID NO: 6) | | | |
| NS2A₃₆₋₄₄ | VAVSFVTLI | D^{b} 9-mer | 997 | 12 |
| | (SEQ ID NO: 7) | | | |
| NS2A₁₄₅₋₁₅₃ | LAVTIMAIL | D^{b} 9-mer | 644 | -- |
| | (SEQ ID NO: 8) | | | |
| NS4B₅₉₋₆₆ | SSVNVSLT | K^{b} 8-mer | 1451 | -- |
| | (SEQ ID NO: 9) | | | |
| NS4B₆₆₋₇₄ | TAIANQATV | D^{b} 9-mer | 1.3 | -- |
| | (SEQ ID NO: 10) | | | |
| NS4B₉₉₋₁₀₇ | YSQVNPITL | D^{b} 9-mer | 1.3 | 1893 |
| | (SEQ ID NO: 11) | | | |
| NS5₂₃₇₋₄₅ | RMLINRFTM | D^{b} 9-mer | 21 | -- |
| | (SEQ ID NO: 12) | | | |

-- An IC 50 > 2000 nM.

### Example 3

### This Example Shows Confirmation of DENV2-Derived CD8⁺ Epitope Identification by Intracellular Cytokine Staining.

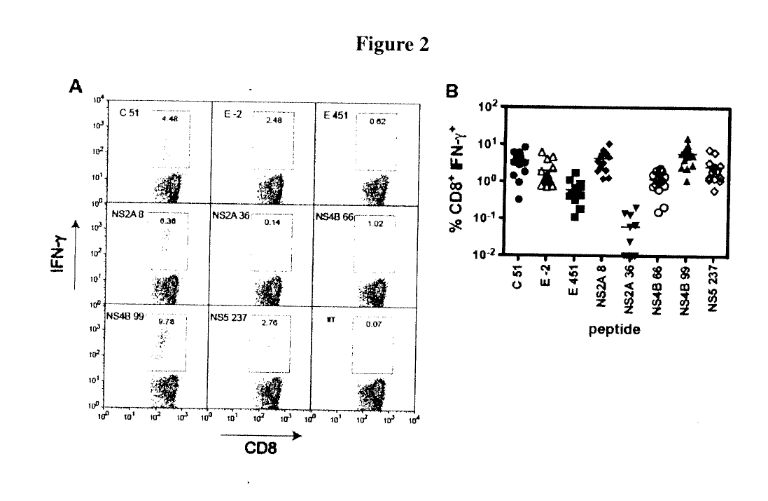

To confirm that the IFN-γ response to the MHC class I-binding DENV peptides was mediated by CD8⁺ T cells, intracellular cytokine staining (ICS) on CD8⁺ T cells isolated from S221-infected wild-type mice was performed. Splenocytes were harvested from C57BL/6 mice 7 days after infection with 10¹¹ genomic equivlalents (GE) S221 and were re-stimulated in vitro with individual DENV2 peptides or an irrelevant peptide (irr) at 0.1 μg/ml in the presence of brefeldin A for 5 h. Cells were then stained for surface CD8 and intracellular IFN-γ and analyzed by flow cytometry. Referring to FIG. 2A, which shows IFN-γ production by DENV2-specific CD8⁺ T cells (representative of four independent studies). The percent of CD8⁺ T cells producing IFN-γ is indicated. Referring to FIG. 2B, the figure is a summary of the DENV2-specific CD8⁺ IFN-γ⁺ response. The response to the irrelevant control peptide was subtracted from the responses to each DENV2 peptide. Each symbol represents one mouse and the bar represents the geometric mean. The identification of the 12 positive peptides for S221 was confirmed by the intracellular cytokine staining (ICS) (FIG. 2A), and the response to the 8 most dominant peptides was determined (FIG. 2B). The responses ranged from 0.14% recognizing NS2A 36 to 9.8% of CD8⁺ T cells recognizing NS4B 99.

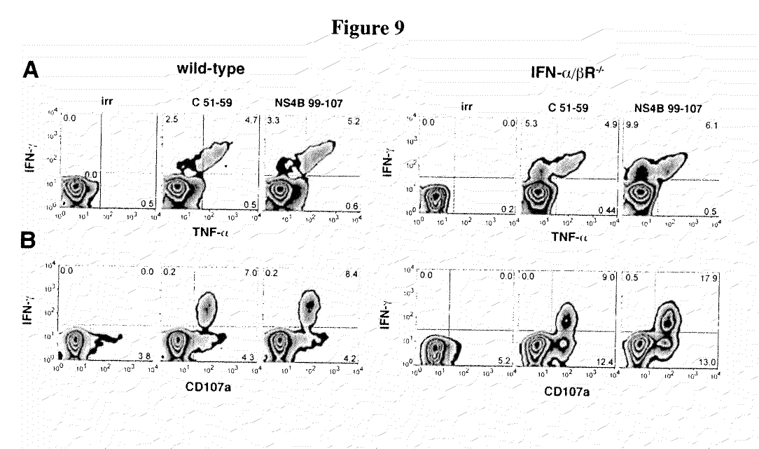

Subsequent studies indicate the number of CD8⁺ T cells producing IFN-γ in response to the seven most dominant epitopes in wild-type mice (FIG. 9A). The responses ranged from an average of 3.3×10⁴ CD8⁺ T cells recognizing E₄₅₁₋₄₅₈ to 6.3×10⁵ cells specific for NS4B₉₉₋₁₀₇ (which corresponds to 0.6% and 5.9% of splenic CD8⁺ T cells, respectively). Responses against the remaining five epitopes were not reproducible above background levels, which is consistent with the greater sensitivity of the ELISPOT assay compared with ICS (Yewdell et al., Immunity 25:533 (2006)). Thus, a set of 12 H-2^{b}-restricted CD8⁺ T cell epitopes derived from DENV were identified, seven of which induce responses detectable by ICS.

As described in Example 2, mapping of the specificity of the response to the DENV2 strains PL046 and the mouse-passaged S221 in C57BL/6 mice identified 12 epitopes from 6 of the 10 DENV proteins. The specificity of the responses to both viruses was the same, although the mouse-passaged S221 induced a more robust CD8⁺ T cell response, due to its increased virulence in mice. In general, the positive peptides were good MHC class I binders, although the immunodominance hierarchy did not correlate exactly with in vitro binding. Other factors, such as T cell precursor frequency and antigen processing during infection, likely affect the immunodominance. Considering the small size of the DENV genome, the CD8⁺ T cell response is relatively broad, and is similar to what has been observed for other small viruses. For example, 16 CD8⁺ T cell epitopes were identified in influenza A-infected C57BL/6 mice (Zhong et al., J Biol Chem 278:45135 (2003)). Not surprisingly, the CD8⁺ T cell response against larger viruses is more broad: 24 murine cytomegalovirus (MCMV)-derived and 49 VACV-derived CD8⁺ T cell epitopes have been identified in C57BL/6 mice (Moutaftsi et al., Nat Biotechnol 24:817 (2006); Munks et al., J Immunol 176:3760 (2006)). However, CD8⁺ T cell responses are generally dominated by a small number of epitopes. Consistent with this, the response to the four most dominant epitopes (C₅₁, NS2A₈, NS4B₉₉, and N55₂₃₇) accounted for 80% of the DENV2-specific response, with the response to the most dominant epitope, NS4B 99 accounting for 30% of the anti-DENV2 response.

### Example 4

This Example Shows Data Indicating that CD8⁺ T Cell Activation is Induced by DENV2.

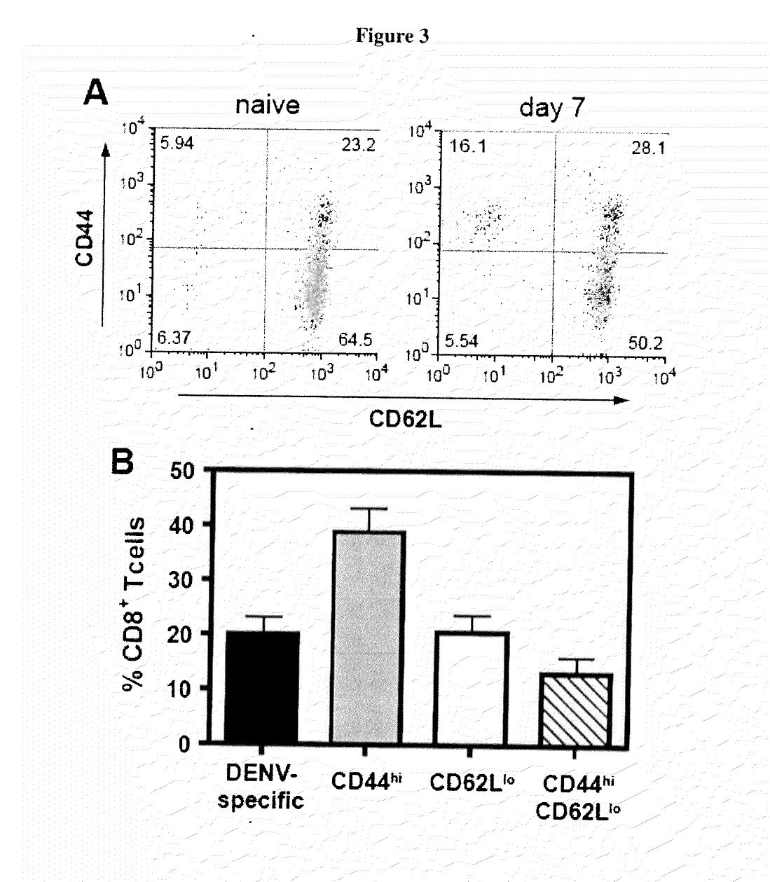

Splenocytes were harvested from naïve C57BL/6 mice or mice 7 days after infection with 10¹¹ GE of S221. The percent of CD8⁺ T cells expressing CD44 and CD62L was determined in four independent studies (FIG. 3A). On day 7 after infection, splenocytes were stimulated with the eight major DENV2 peptides (individually; at 0.1 μg/ml) in the presence of brefeldin A, and stained for surface CD8 and intracellular IFN-γ. The percent of CD8⁺ T cells producing IFN-γ in response to the irrelevant control peptide was subtracted from the response to each DENV2 peptide to determine the net response. The net responses to the eight major DENV2 peptides were added to determine the percent of DENV2-specific CD44^{hi}, CD62^{lo}, and CD44^{hi}CD62L^{lo} CD8⁺ T cells (FIG. 3B). The data are expressed as the mean percent±SEM of 11-14 mice tested in at least four independent experiments.

S221 infection of C57BL/6 mice did not result in an increase in splenic CD8⁺ T cells numbers, but did induce CD8⁺ T cell activation, as measured by upregulation of CD44 and downregulation of CD62L on day 7 after infection (FIG. 3A). Approximately 40% of the CD8⁺ T cells were CD44^{hi}, 20% CD62L^{lo}, and 13% were CD44^{hi}CD62L^{lo} effector cells (FIG. 3B). UV-inactivated S221 did not induce T cell activation (data not shown). In an attempt to estimate the percentage of CD8⁺ T cells in S221-infected mice that are antigen-specific, the net response to the eight dominant epitopes was added, and found to be 20% (FIG. 3B). Compared with the percentage of CD62L^{lo} or CD44^{hi}CD62L^{lo}CD8⁺ T cells, the DENV2-specific response was equivalent or higher, suggesting the majority, if not all, of the activated CD8⁺ T cells were DENV2-specific. Although the percentage of CD44^{hi}CD8⁺ T cells was double the percent of DENV2-specific CD8⁺ T cells, it was found that approximately 20-25% of CD8⁺ T cells in naïve mice were CD44^{hi}. Taken together, these data indicate that antigen-specific CD8⁺ T cells account for the majority of activated CD8⁺ T cells in S221-infected C57BL/6 mice.

### Example 5

### This Example Shows Development of DENV Strain, S221 and Contribution of CD8⁺ T Cells to Recognition of 8 Major DENV2 CD8⁺ T Cell Epitopes in IFN-α/βR^{-/-} Mice.

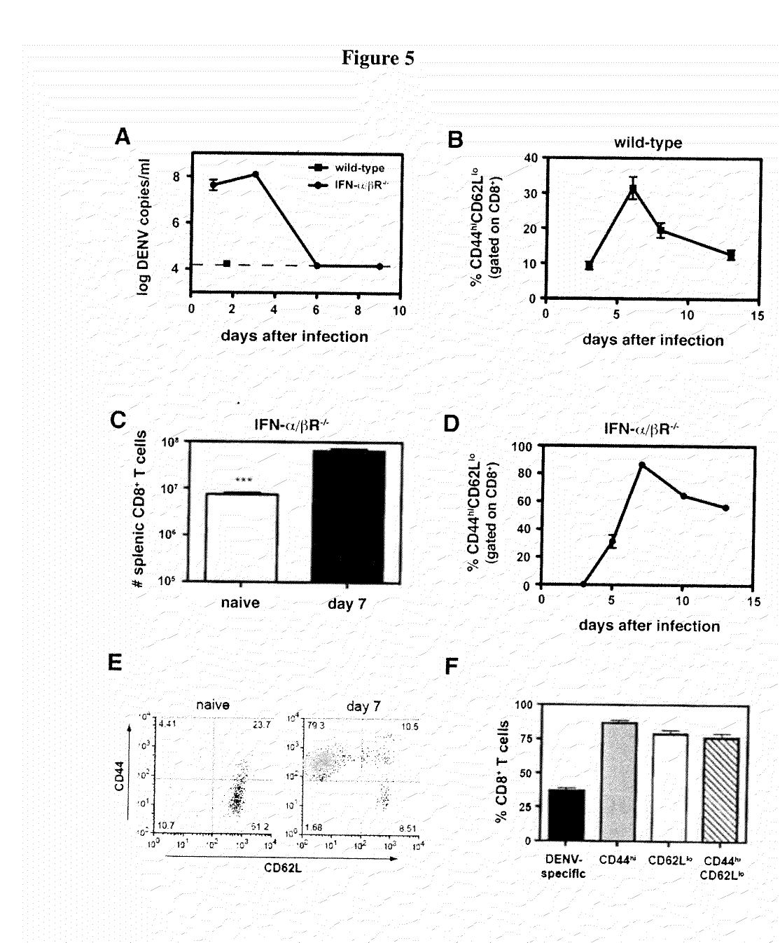

Recently a novel DENV2 strain, D2S10, was isolated which is reportedly more virulent in mice genetically deficient, in both the IFN-α/βR and IFN-γR than the parental strain, PL046 Shresta et al., J Virol 80:10208 (2006). A biological clone of D2S10, termed S221, was isolated by triple plaque-purification. Wild-type C57BL/6 mice infected with S221 did not manifest any symptoms of disease, and the virus was undetectable by real-time polymerase chain reaction (RT-PCR) in the serum 40 hours after infection (FIG. 5A). Thus, to study the contribution of CD8⁺ T cells in protection against DENV infection a mouse model was developed in which the virus replicates at detectable levels. Given the importance of the IFN system in most antiviral responses (Muller et al., Science 264:1918 (1994); van den Broek et al., J Virol 69:4792 (1995)), including the anti-DENV response (Diamond et al., J Virol 74:4957 (2000)), the susceptibility of IFN-α/βR^{-/-} C57BL/6 mice to the S221 virus was studied. Infection of IFN-α/βR^{-/-} mice with S221 resulted in a productive infection, and the mice showed symptoms of disease, including hunched posture and ruffled fur at the peak of viremia, between 2 and 4 days after infection (FIG. 5A). The mice subsequently recovered and cleared the virus from the blood by approximately day 6.

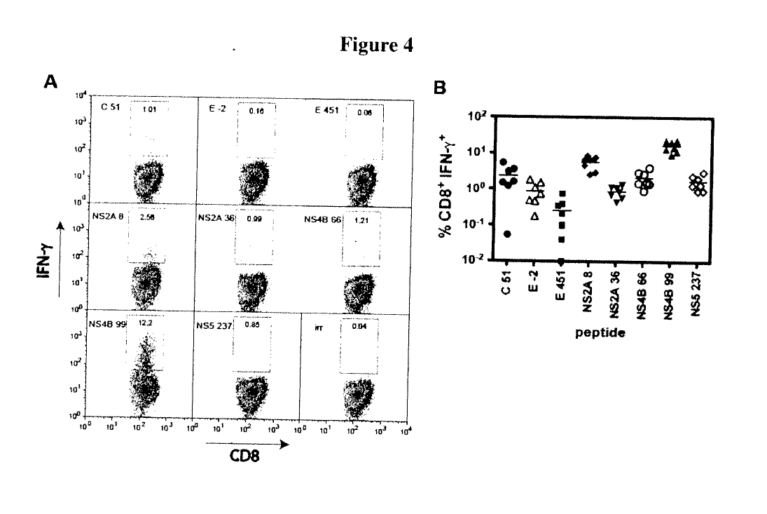

The S221-specific CD8⁺ T cell response in the IFN-α/βR^{-/-} mice was studied. Splenocytes harvested from IFN-α/βR^{-/-} mice 7 days after infection with 10¹⁰ GE S221 were re-stimulated in vitro with individual DENV2 peptides (or an irrelevant peptide) at 0.1 μg/ml in the presence of brefeldin A for 5 h; and then stained for CD8 and intracellular IFN-γ. The percent of CD8⁺ T cells producing IFN-γ is indicated in FIG. 4A (results representative of three independent studies). Referring to FIG. 4B, the figure shows a summary of the DENV2-specific CD8⁺ IFN-γ⁺ response. The response to the irrelevant peptide was subtracted from the response to each DENV2 peptide. Each symbol represents one mouse and the bar represents the geometric mean. Results are derived from three independent studies.

The 8 DENV2 epitopes identified in wild-type C57BL/6 mice were also recognized in the IFN-α/βR^{-/-} mice (FIGS. 4A and 4B). Results show that the immunodominance hierarchy was somewhat altered compared with wild-type mice, for example, the response to NS2A 36 was higher than the response to E₄₅₁ in the IFN-α/βR^{-/-}, but not wild-type mice. However, overall the response was similar and the dominant epitopes were the same in both genotypes.

S221 infection of IFN-α/βR^{-/-} mice is the most relevant mouse model developed to date for studying the adaptive immune response to DENV. A variety of mouse models for DENV infection have been developed, but most are not ideal for investigating DENV pathogenesis. For example, these models include intracerebral route of infection, infection with mouse brain-adapted DENV strains that predominantly target the central nervous system, chimeric mice transplanted with human cells, and severely immunocompromised mice. A mouse model of DENV2 infection that mimics key aspects of DHF/DSS was previously reported; specifically, infection of IFN-α/βR^{-/-}×IFN-γR^{-/-} mice with the DENV2 strain, D2S10, resulted in vascular leakage and an early, TNF-α-mediated death (Shresta et al., J Virol 80:10208 (2006)). However, S221-infected IFN-α/βR^{-/-} mice even more closely resembles human DENV infections by demonstrating high viremia and eventual clearance. The use of IFN-α/βR^{-/-} mice is also advantageous in that the mice are less immunocompromised than the IFN-α/βR^{-/-}×IFN-γR^{-/-} mice, and allowed us to investigate the anti-DENV IFN-γ response. Further, the results showed CD8⁺ T cell IFN-γ response is IFN-α/β-independent. It is likely IL-12 compensates for the lack of IFN-α/β in inducing IFN-γ.

A caveat to using the IFN-α/βR^{-/-} mice can be that IFN-α/β has been found to act directly on CD8⁺ T cells and affect their expansion and memory formation after LCMV infection (Kolumam et al., J Exp Med 202:637 (2005)). However, the dependence on IFN-α/β signaling is pathogen-specific, as one study found the lack of IFN-α/β signaling significantly impaired CD8⁺ T cell expansion in response to LCMV, but had only a minimal effect during infections with VACV, vesicular stomatitis virus, or Listeria monocytogenes (Thompson et al., J Immunol 177:1746 (2006)). In contrast, another group found LCMV-induced CD8⁺ T cell proliferation and IFN-γ production was IFN-α/β-independent (Cousens et al., J Exp Med 189:1315 (1999)). The anti-DENV CD8⁺ T cell response appears to be IFN-α/β-independent, as the IFN-α/βR^{-/-} mice initiated a very robust anti-DENV CD8⁺ T cell response. CD8⁺ T cell expansion, activation, and antigen-specific IFN-γ production (based on CD8⁺ T cell number) were all greater in the knockout mice compared with wild-type mice, likely due to the enhanced viral replication. Furthermore, the memory response to DENV does not appear to be impaired in IFN-α/βR^{-/-} mice, as the results demonstrated a similar percentage of DENV2-specific T cells in knockout and wild-type mice 40 days after infection. Direct comparison of the magnitude of the anti-S221 CD8⁺ T cell response between wild-type and knockout mice is difficult due to the differences in viral replication, yet the data suggest the anti-DENV CD8⁺ T cell response is not impaired in the IFN-α/βR^{-/-} mice.

### Example 6

This Example Includes Data Indicating that CD8⁺ T Cell Activation is Induced in Wild-Type and IFN-α/βR^{-/-} C57BL/6 Mice by DENV2.

To determine the CD8⁺ T cell response to S221, the number and activation state of splenic and blood CD8⁺ T cells in wild-type and IFN-α/βR^{-/-} mice infected with DENV was studied. Referring to FIGS. 5A to 5D, DENV infection results in a CD8⁺ T cell response in wild-type and IFN-α/βR^{-/-} mice, but detectable levels of viremia only in IFN-α/βR^{-/-} mice. Wild-type mice (n=3) infected with 10¹¹ GE of S221 and IFN-α/βR^{-/-} mice (n=6) infected with 10¹⁰ GE were bled and the DENV RNA levels in the serum measured by real-time RT-PCR (FIG. 5A). The dashed line indicates the limit of detection. Blood lymphocytes were obtained from wild-type mice (n=4) on days 3, 6, 8, and 13 after infection with 10¹¹ GE of S221 (FIG. 5B). The percentage of CD44^{hi}CD62L^{lo} cells (gated on CD8⁺ T cells) is indicated. Numbers of splenic CD8⁺ T cells in naïve IFN-α/βR^{-/-} mice (n=4) and IFN-α/βR^{-/-} mice infected with 10¹⁰ GE of S221 (n=7) were determined (FIG. 5C), ***p<0.0001 for naïve versus infected mice. Blood lymphocytes were obtained from IFN-a/bR^{-/-} mice (n=3) on days 3, 5, 7, 10, and 13 after infection with 10¹⁰ GE of S221. The percentage of CD44^{hi}CD62L^{lo} cells (gated on CD8⁺ T cells) is shown in FIG. 5D.

In wild-type mice, infection with S221 did not lead to an increase in splenic CD8⁺ T cell numbers; however, the CD8⁺ T cells were activated, as measured by upregulation of CD44 and downregulation of CD62L on CD8⁺ T cells. In the spleen, 13% of CD8⁺ T cells were CD44^{hi}CD62L^{lo} on day 7 after infection (13±3%, mean±SEM, n=11). In the blood, activation peaked at day 6, with approximately 30% of the CD8⁺ T cells activated (FIG. 5B). UV-inactivated S221 did not induce CD8⁺ T cell activation, indicating that DENV replication or at least input strand translation is required for T cell activation.

In S221-infected IFN-α/βR^{-/-} mice a 9-fold increase in splenic CD8⁺ T cell numbers was observed (FIG. 5C). Most of the splenic CD8⁺ T cells were CD44^{hi}CD62L^{lo} on day 7 (76±3%, n=7). Activation of circulating CD8⁺ T cells peaked at approximately day 7, with 87% of CD8⁺ T cells activated (FIG. 5D).

Taken together, these data demonstrate that DENV induces CD8⁺ T cell activation in both wild-type and IFN-α/βR^{-/-} mice. As expected, a greater percentage of CD8⁺ T cells were activated in the IFN-α/βR^{-/-} mice than in wild-type mice, and CD8⁺ T cell proliferation was observed in the IFN-α/βR^{-/-} mice but not wild-type mice, likely due to increased DENV replication.

The S221-specific CD8⁺ T cell response in IFN-α/βR^{-/-} mice was further studied. Splenocytes were harvested from naïve IFN-α/βR^{-/-} mice or mice 7 days after infection with 10¹⁰ GE of S221. Referring to FIGS. 5C, 5E and 5F, the number of splenic CD8⁺ T cells in naïve (n=4) and infected (n=7) mice was measured (results described above). Expression of CD44 and CD62L on CD8⁺ T cells from naïve and infected mice is shown in FIG. 5E. On day 7 after infection, splenocytes were stimulated with eight of the major DENV2 peptides (individually; at 0.1 ng/ml) in the presence of brefeldin A, and stained for surface CD8 and intracellular IFN-γ. The percent of CD8⁺ T cells producing IFN-γ in response to the irrelevant control peptide was subtracted from the response to each DENV2 peptide to determine the net response, as shown in FIG. 5F. The net responses to eight of the major DENV2 peptides were added to determine the percent of DENV2-specific CD8⁺ T cells. The percent of CD44^{hi}, CD62^{lo}, and CD44^{hi}CD62L^{lo}CD8⁺ T cells in also shown in FIG. 5F. The data are expressed as the percent±SEM of seven mice tested in three independent experiments.

Infection of IFN-α/βR^{-/-} mice with S221 results in a large, approximately 9-fold, expansion of splenic CD8⁺ T cells (FIG. 5C). Most of the CD8⁺ T cells were activated in these mice, based on upregulation of CD44 and downregulation of CD62L (FIG. 5E). Approximately 35% of the CD8⁺ T cell response was DENV2-specific (the sum of the net response to the eight major epitopes), whereas approximately 75% of the splenic CD8⁺ T cells were CD44^{hi}TD62L^{lo} (FIG. 5F). This indicates the remaining 40% of the activated CD8⁺ T cells were specific for other epitopes, or that there was bystander activation of CD8⁺ T cells in these mice.

Activation marker expression has been used to quantitate the magnitude of antigen-specific responses to other pathogens (Kotturi et al., J Virol 81:4928 (2007); Munks et al., J Immunol 176:3760 (2006); Masopust et al., J Virol 81:2002 (2007)). Studies of LCMV, VACV and MCMV have found the vast majority of CD8⁺ T cell responses are antigen-specific, and bystander activation does not play a significant role (Kotturi et al., J Virol 81:4928 (2007); Moutaftsi et al., Nat Biotechnol 24:817 (2006); Munks et al., J Immunol 176:3760 (2006); Masopust et al., J Virol 81:2002 (2007)). In agreement with those studies, the results indicate the percent of CD8⁺ T cells specific for the eight major DENV2 epitopes in C57BL/6 mice was equivalent to the percent of activated cells, indicating the entire response is DENV2-specific. In addition, these data suggest that most, if not all, of the DENV2-derived epitopes recognized in C57BL/6 mice have been identified. In contrast, the responses to the eight epitopes accounted for less than half of the total CD8⁺ T cell response in IFN-α/βR^{-/-} mice, suggesting other epitopes contribute to the response, and/or there was bystander activation of CD8⁺ T cells non specific for DENV2. One epitope in particular (M₆₀) was identified that is a dominant epitope in IFN-α/βR^{-/-} mice (recognized by 11% of CD8⁺ T cells), but not C57BL/6 mice (0.4%). It is likely the response to other epitopes is enhanced in the knockout mice compared with the wild-type mice, due to the much larger CD8⁺ T cell response.

### Example 7

This Example Shows that CD8⁺ Cell-Depletion Impairs DENV Clearance.

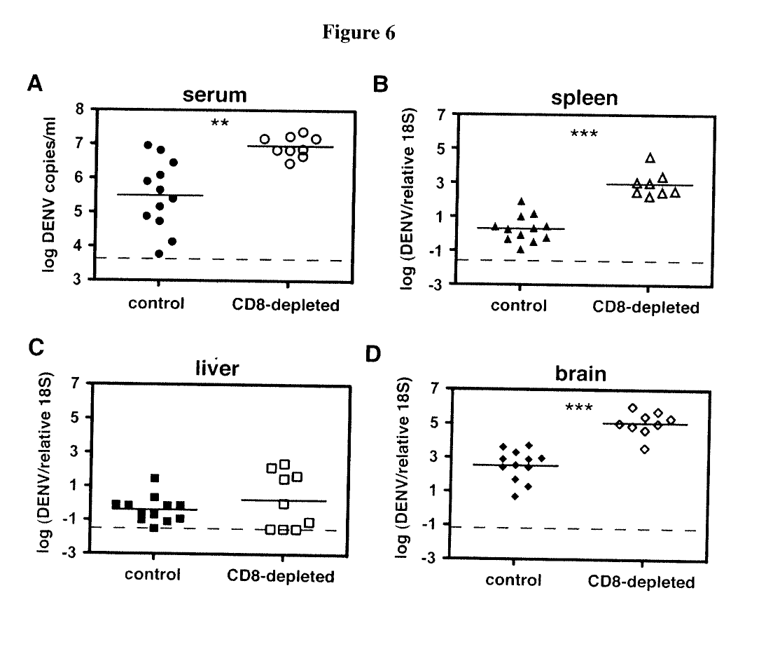

As IFN-α/βR^{-/-} mice mount a robust CD8⁺ T cell response following DENV infection, a study was performed to define the contribution of these cells to controlling infection. IFN-α/βR^{-/-} mice were CD8⁺ T cell-depleted prior to infection with S221, and virus levels were measured in the serum, spleen, liver, and brain by real-time polymerase chain reaction (RT-PCR) 3 and 6 days after infection. In the study, IFN-α/βR^{-/-} mice were depleted of CD8⁺ T cells by administration of an anti-CD8 Ab (or given an isotype control Ab) 3 days and 1 day before infection with 10¹¹ GE of S221. Mice were sacrificed 6 days later, and DENV RNA levels in the serum, spleen, liver and brain were quantified by real-time RT-PCR. FIGS. 6A-6D show depletion of CD8⁺ T cells prior to DENV infection results in increased viral loads. Data are expressed as DENV copies per ml of sera, or DENV units normalized to 18S rRNA levels for the spleen, liver, and brain. Each symbol represents one mouse, the bar represents the geometric mean, and the dashed line is the limit of detection, ** p<0.001 for serum, ***p<0.0001 for spleen and brain; and p=0.39 for viral load in the liver of CD8-depleted mice compared with control mice.

On day 3 after infection all of the mice appeared hunched and ruffled, and DENV levels in the serum, spleen, liver, and brain were similar in control and CD8-depleted mice. In contrast, by day 6 after infection, the control mice appeared healthy, whereas the CD8-depleted mice were still hunched and ruffled, and some exhibited hind limb weakness. CD8-depleted mice had significantly higher DENV RNA levels than control mice in the serum, spleen, and brain, (30-, 460-, and 350-fold, respectively) but no differences were observed in the liver (FIG. 6). DENV RNA levels in the control mice declined from day 3 to day 6, whereas viral RNA levels in the brain of CD8-depleted mice increased from day 3 to day 6. These results demonstrate that CD8⁺ T cells control DENV infection in IFN-α/βR^{-/-} mice.

Although CD8⁺ T cells have been reported to be important in the host response to West Nile virus (WNV) (Shrestha et al., J Virol 78:8312 (2004)), a protective role in DENV infection has not been demonstrated and studies to date have mainly focused on the potentially pathogenic role of T cells. Determining the contribution of CD8⁺ T cells to protection is important in designing a DENV vaccine, and these data demonstrate that CD8⁺ T cells contribute to protection during primary DENV infection. The results of the study show that depletion of CD8⁺ T cells resulted in an impaired ability of the mice to clear DENV and significantly higher viral loads in the serum, spleen, and brain (FIG. 6).

Nervous system involvement in human DENV infection is rare (Patey et al., Am J Trop Med Hyg 48:793 (1993)), but it is a major target in mouse models of DENV infection (Lin et al., J Virol 72:9729 (1998); Johnson et al., J. Virol 73:783 (1999)). Whether CD8⁺ T cells play a role in preventing DENV dissemination to the brain or limiting viral replication in the brain is as yet unknown. Although no difference in DENV levels in the liver between control and depleted mice was observed, the viral load was low in both groups, and the liver therefore does not appear to be a major target of DENV in this mouse model. Altogether, the data show that in the absence of IFN-α/β signaling, CD8⁺ T cells contribute to controlling DENV infection.

### Example 8

This Example Includes Data Showing Depletion of CD4⁺ and/or CD8⁺ T Cells Prior to DENV Infection Results in Increased Viral Load.

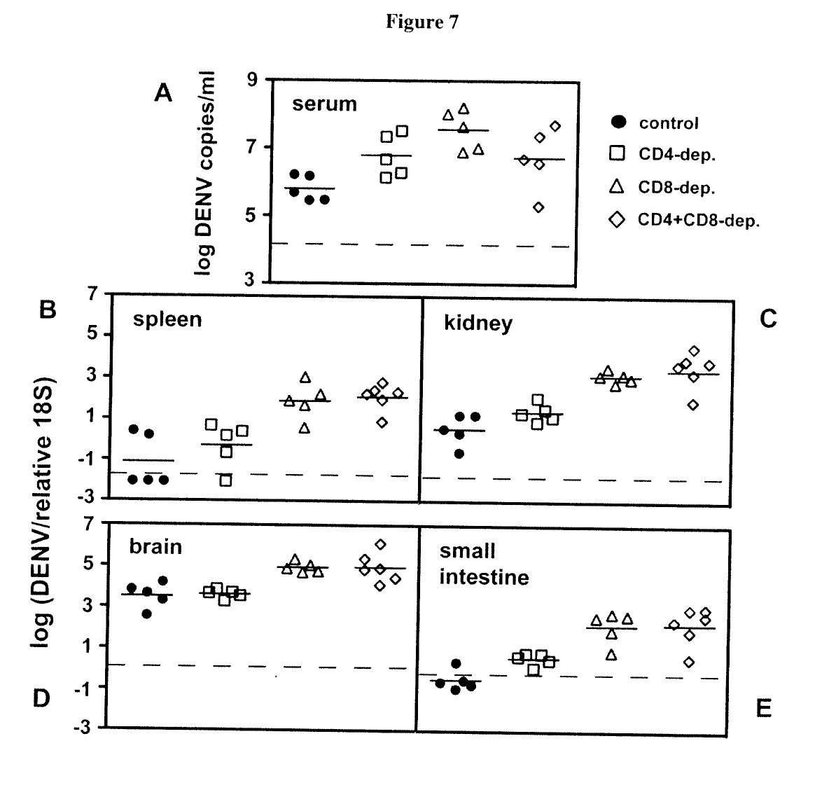

IFN-α/βR^{-/-} mice were depleted of CD4⁺ and/or CD8⁺ T cells by intraperitoneal (i.p.) administration of anti-CD4 Ab (GK1.5, 250 μg) and/or anti-CD8 Ab (2.43, 250 μg) or given an isotype control Ab 3 days and 1 day before intravenous (i.v.) infection with 10¹¹ GE of the DENV2 strain, S221. The mice were sacrificed 6 days later, and DENV RNA levels in the serum, spleen, kidney, small intestine and brain were quantified by real-time RT-PCR, the results are shown in FIGS. 7A-E. Data are expressed as DENV copies per ml of sera, or DENV units normalized to 18S rRNA levels for the tissues. Each symbol represents one mouse, the bar represents the geometric mean, and the dashed line is the limit of detection.

These data demonstrate that CD4⁺ T cells also contribute to controlling DENV infection in IFN-α/βR^{-/-} mice, as mice depleted of CD4⁺ T cells had higher DENV RNA levels in the serum, kidney, spleen, and small intestine. However, these data indicate that the relative contribution of CD8⁺ T cells to the anti-DENV immune response is greater than that of CD4⁺ T cells.

### Example 9

This Example Includes Data Indicating that the Identified Epitopes are Recognized in IFN-α/βR^{-/-} Mice.

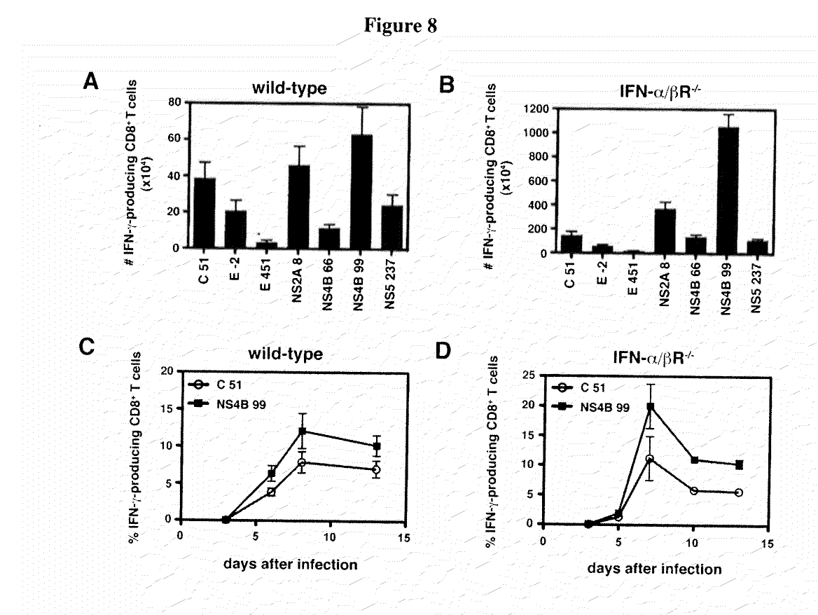

The initial epitope identification studies were performed in wild-type mice. Because IFN-α/βR^{-/-} mice were used to study the contribution of CD8⁺ T cells to controlling DENV infection, the specificity of the CD8⁺ T cell response was examined in these mice. Confirmation of DENV-derived CD8⁺ T cell epitope identification in wild-type and IFN-α/βR^{-/-} mice was performed by intracellular cytokine staining (ICS) (FIGS. 8A-10D). Referring to FIGS. 8A and 8B, splenocytes harvested from wild-type (A) or IFN-α/βR^{-/-} (B) mice 7 days after infection with 10¹¹ or 10¹⁰ GE of S221, respectively, were re-stimulated in vitro with individual DENV peptides or an irrelevant peptide. Cells were then stained for surface CD8 and intracellular IFN-γ and analyzed by flow cytometry. The response to the irrelevant peptide was subtracted from the response to each DENV peptide, and the number of CD8⁺ T cells producing IFN-y is indicated. Results are expressed as the mean±SEM of 13 wild-type and 7 IFN-α/βR^{-/-} mice were tested in at least three independent experiments. Referring FIGS. 8C and D, the figures show kinetics of the DENV-specific CD8⁺ T cell response as determined by the study. Wild-type mice ((C), n=4) and IFN-α/βR^{-/-} mice ((D), n=3) were infected with 10¹¹ or 10¹⁰ GE of S221, respectively, and blood lymphocytes were isolated at various time points. Stimulation and ICS were performed as in (A) and (B), and the percentage of CD8⁺ T cells producing IFN-γ is shown.

As described above, the responses to the seven dominant DENV epitopes identified in wild-type mice were examined in wild type and IFN-α/βR^{-/-} mice by ICS (FIGS. 8A and 8B). The responses ranged from an average of 1.6×10⁵ CD8⁺ T cells specific for E₄₅₁₋₄₅₈ to 1.1×10⁷ recognizing NS4B₉₉₋₁₀₇ (0.3% and 16.5% of splenic CD8⁺ T cells, respectively). The specificity of the response was similar in the IFN-α/βR^{-/-} and wild-type mice, with C₅₁₋₅₉, NS2A₈₋₁₅, and NS4B₉₉₋₁₀₇ dominating. However, the immunodominance hierarchies were not identical; for example, the response to NS4B₆₆₋₇₄ was higher than the response to E₂₋₆ in the IFN-α/βR^{-/-} but not wild-type mice. In addition, the magnitude of the response differed, with a greater number of CD8⁺ T cells specific for each epitope in the IFN-α/βR^{-/-} mice. Finally, kinetic analysis revealed that the DENV-specific response of circulating CD8⁺ T cells peaked at approximately day 7/8 in both the wild-type and IFN-α/βR^{-/-} mice (FIGS. 8C and 8D).

Results from this study and preceding Examples show that the specificity of the CD8⁺ T cell response was similar in the wild-type and IFN-α/βR^{-/-} mice. All the epitopes identified in wild-type mice were also positive in IFN-α/βR^{-/-} mice, and the immunodominance hierarchy of the seven dominant epitopes was similar. Similar to these findings, the IFN-α/βR^{-/-} mice had a higher viral load, which has been shown to affect the immunodominance of the anti-LCMV CD8⁺ T cell response (Probst et al., J Immunol 171:5415 (2003)). However, the difference in DENV replication between wild-type and IFN-α/βR^{-/-} mice did not significantly alter the hierarchy of the seven dominant epitopes. Overall, the results suggest that lack of IFN-α/β signaling does not appreciably affect the immunodominance of the CD8⁺ T cell response to DENV. This finding, combined with the high viremia and CD8⁺ T cell activation observed in S221-infected IFN-α/βR^{-/-} mice supports the use of this model to study the role of CD8⁺ T cells during DENV infection.

Two DENV-derived CD8⁺ T cell epitopes (NS3₂₉₈₋₃₀₆ and E₃₃₁₋₃₃₉) were previously identified in mice by isolating T cell clones from DENV-immunized BALB/c (H-2^{d}) mice (Rothman et al, J Virol 70:6540 (1996); Spaulding et al., J Virol 73:398 (1999)). However, the studies herein use a predictive approach spanning the entire proteome to map the CD8⁺ T cell response to DENV. A total of 12 epitopes derived from 6 of the 10 DENV proteins were identified in wild-type C57BL/6 mice. In general, the positive peptides exhibited intermediate or high binding affinity to MHC class I in vitro. As expected, the immunodominance hierarchy did not strictly correlate with in vitro binding affinities, as other factors, such as T cell precursor frequency and antigen processing during infection affect immunodominance (Yewdell et al., Immunity 25:533 (2006)). Considering the small size of the DENV genome, this CD8⁺ T cell response is relatively broad, and is similar to the 16 CD8⁺ T cell epitopes identified in influenza A-infected C57BL/6 mice (Zhong et al., J Biol Chem 278:45135 (2003)). Broad CD8⁺ T cell responses in C57BL/6 mice have been identified for LCMV (28 epitopes) (Kotturi et al., J Virol 81:4928 (2007)), and for larger viruses such as murine cytomegalovirus (24 epitopes) (Munks et al., J Immunol 176:3760 (2006)) and VACV (49 epitopes) (Moutaftsi et al., Nat Biotechnol 24:817 (2006).

A number of DENV-derived CD8⁺ T cell epitopes have been identified in humans, including epitopes from the E, NS3, NS4A, NS4B, and NS5 proteins (Mongkolsapaya et al., Nat Med 9:921 (2003); Imrie et al., J Virol 81:10081 (2007); Bashyam et al., J Immunol 176:2817 (2006); Simmons et al., J Virol 79:5665 (2005); Zivny et al., J Immunol 163:2754 (1999); Mathew et al., J Virol 72:3999 (1998). Based on the isolation of a number of NS3-specific CD8⁺ T cell clones, this protein has been postulated as a major target of the T cell response (Rothman et al., J Clin Invest 113:946 (2004)). However, this study did not identify any immunogenic NS3 epitopes, even though seven NS3 peptides were predicted and tested by IFN-γ ELISPOT, which could be due to species-specific differences in the CD8⁺ T cell responses.

### Example 10

This Example Shows Data Indicating DENV-Specific CD8⁺ T have a Polyfunctional Phenotype.

Because polyfunctional CD8⁺ T cell responses are associated with protection (Harari et al., Immunol Rev 211:236 (2006)), the phenotype of the DENV-specific CD8⁺ T cells in wild-type and IFN-α/βR^{-/-} mice was studied in more detail by measuring CD107a expression (CD107a, or LAMP-1, is expressed on the cell surface upon degranulation) and TNF-α production for two of the DENV-specific CD8⁺ T cell populations, as described above. To collect the data, splenocytes harvested from wild-type or IFN-α/βR^{-/-} mice 7 days after infection with 10¹¹ or 10¹⁰ GE of S221, respectively, were re-stimulated in vitro with C₅₁₋₅₉, NS4B₉₉₋₁₀₇, or an irrelevant peptide. An anti-CD107a Ab was added for the duration of the stimulation. Cells were then stained for surface CD8, intracellular IFN-γ and TNF-α, and analyzed by flow cytometry. Representative density plots are shown in FIGS. 9A and 9B.

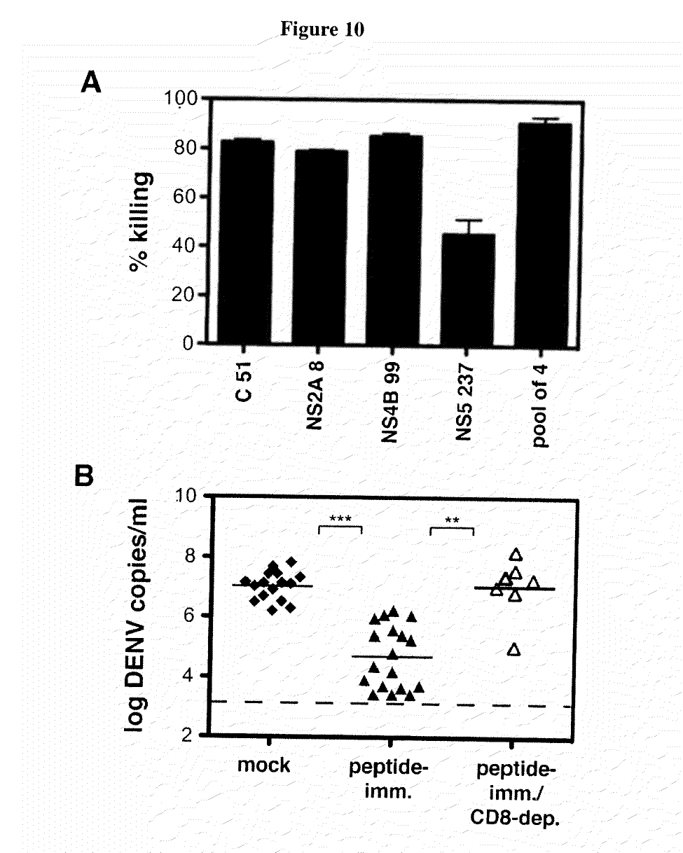

The CD8⁺ T cells recognizing C₅₁₋₅₉ and NS4B₉₉₋₁₀₇ produced TNF-α (FIG. 9A) and expressed CD107a on the surface (FIG. 10B). Not all of the IFN-γ⁺ cells also produced TNF-α, but all of the IFN-γ⁺ cells were CD107a⁺. The CD107a⁺ cells were IFN-γ⁻/TNF-α⁻, IFN-γ⁺/TNF-α⁺, or IFN-γ⁺/TNF-γ⁺ (data not shown). Thus, the specificity of the CD8⁺ T cell response is similar in wild-type and IFN-α/βR^{-/-} mice, with DENV-specific CD8⁺ T cells producing IFN-γ, TNF-α, and degranulating. Together with the results in FIG. 5, the data reveal that the CD8⁺ T cell response to DENV is polyfunctional, and that the response, including proliferation, degranulation, and cytokine production, can develop in the absence of IFN-α/β signaling.

Results from this study and the preceding examples indicate that the CD8⁺ T cell response to primary DENV infection does not require signaling through the IFN-α/βR^{-/-}. CD8⁺ T cell expansion, activation, and antigen-specific IFN-g production (based on CD8⁺ T cell number) were enhanced in IFN-α/βR^{-/-} mice compared with wild-type mice (FIGS. 4 and 5), likely a result of increased viral replication. In addition, the percentages of TNF-α or CD107a⁺ CD8⁺ T cells were similar or greater in the IFN-α/βR^{-/-} mice (FIG. 9). Thus, in terms of proliferation, cytokine production, and degranulation, the CD8⁺ T cell response to DENV does not require type I IFNs. It is probable that IL-2 and IL-12 compensate for the lack of IFN-α/βR^{-/-} signaling in inducing CD8⁺ T cell proliferation and IFN-g production, respectively.

### Example 11

This Example Includes Studies Showing in vivo Killing of DENV Peptide-Pulsed Target Cells.

Having found that DENV-specific CD8⁺ T cells express a degranulation marker, studies were performed to analyze their cytotoxic activity using an in vivo cytotoxicity assay. CFSE-labeled splenocytes pulsed with individual immunodominant DENV peptides (C 51-59, NS2A 8-15, NS4B 99-107, NS5 237-245) or a pool of the four peptides were transferred into DENV-immune IFN-α/βR^{-/-} mice, and the percentage of target cell killing was calculated after 4 hours. To collect the data IFN-α/βR^{-/-} mice infected 7 days previously with 10¹⁰ GE of S221 were injected intravenously (i.v.) with CFSE-labeled target cells pulsed with C₅₁₋₉, NS2A₈₋₁₅, NS4B₉₉₋₁₀₇, NS5₂₃₇₋₂₄₅ or a pool of the four peptides (n=3-6 mice per group). After 4 hours, splenocytes were harvested, analyzed by flow cytometry, and the percentage killing was calculated (FIG. 10A).

Target cells pulsed with the pool of four peptides were efficiently killed (91% killing), and killing of targets pulsed with the individual peptides ranged from 46% for NS5 237-245 to 85% for NS4B 99-107 (FIG. 10A). These results show that DENV-specific CDS⁺ T cells mediate cytotoxicity in DENV-infected IFN-α/βR^{-/-} mice.

In general, antiviral activity of CD8⁺ T cells is mediated by the production of cytokines, including IFN-γ and TNF-α, and direct killing of infected cells. These results and results from the preceding examples show that DENV-specific CD8⁺ T cells produce IFN-γ, TNF-α, and demonstrate potent cytotoxic activity in vivo. As DENV-specific CD8⁺ T cells degranulate, it is likely perforin/granzyme-mediated killing contributes to viral clearance.

### Example 12

This Example Includes Studies Showing Vaccination with DENV CD8⁺ T Cell Epitopes Controls Viral Load.

Since depleting CD8⁺ T cells resulted in increased viral loads and DENV-specific CD8⁺ T cells demonstrated in vivo cytotoxic activity, studies were performed to determine whether enhancing the anti-DENV CD8⁺ T cell response through peptide immunization would contribute to protection against a subsequent DENV challenge. Specifically, the effect of peptide vaccination on viremia was determined by immunizing IFN-α/βR^{-/-} mice with DENV peptides prior to infection with S221 (FIG. 10A). Mice were immunized with four dominant DENV epitopes in an attempt to induce a multispecific T cell response, which is desirable to prevent possible viral escape through mutation (Welsh et al., Nat Rev Microbiol 5:555 (2007)). At day 4 after infection, viremia in the serum was measured by real-time RT-PCR, as described above. The peptide-immunization resulted in enhanced control of DENV infection, with 350-fold lower serum DENV RNA levels in peptide-immunized mice than mock-immunized mice (FIG. 10B). To confirm that the protection was mediated by CD8⁺ T cells, CD8⁺ T cells were depleted from a group of peptide-immunized mice prior to infection, and it was found that this abrogated the protective effect. Thus, the data demonstrate that a preexisting DENV-specific CD8⁺ T cell response induced by peptide vaccination enhances viral clearance.

Most dengue infections are asymptomatic or classified as DF, whereas DHF/DSS accounts for a small percentage of dengue cases, indicating that in most infections the host immune response is protective. These data indicate that CD8⁺ T cells contribute to protection during primary infection by reducing viral load and that CD8⁺ T cells are an important component to a protective immune response. As mentioned above, however, cross-reactive memory T cells have been postulated to contribute to immunopathology during heterologous infections, and the relative contribution of CD8⁺ T cells to protection versus pathogenesis during secondary infections remains to be determined.

This study shows that immunization with four dominant epitopes prior to infection resulted in enhanced DENV clearance, and this protection was mediated by CD8⁺ T cells. These results indicate that vaccination with T cell epitopes can reduce viremia. Current DENV vaccines under development attempt to elicit a neutralizing Ab response, and need to protect against all four serotypes so as to avoid Ab-dependent enhancement. Vaccine research thus far has focused on live attenuated tetravalent vaccines, but subunit and DNA vaccines are in development (Whitehead et al., Nat Rev Microbiol 5:518 (2007)).

A desirable attribute of a DENV vaccine would be the induction of T cell responses to highly conserved epitopes that will protect against all four serotypes (Khan et al., Cell Immunol 244:141 (2006)). Based on the original antigenic sin hypothesis, vaccination with variable CD8⁺ T cell epitopes could lead to pathology by activating cross-reactive memory T cells that respond aberrantly, and should therefore be avoided. The non-structural proteins of DENV, and NS3, NS4B, and NS5 in particular, are highly conserved across serotypes, and likely contain good vaccine candidates.

Results from the Examples described herein reveal a critical role for CD8⁺ T cells in the immune response to an important human pathogen, and provide a rationale for the inclusion of CD8⁺ T cell epitopes in DENV vaccines. Furthermore, identification of the CD8⁺ T cell epitopes recognized during DENV infection in combination with a new mouse model can provide the foundation for elucidating the protective versus pathogenic role of CD8⁺ T cells during secondary infections.

### Example 13

### This Example Shows the Identification of DENV2-Derived Human HLA A*0201 Epitopes.

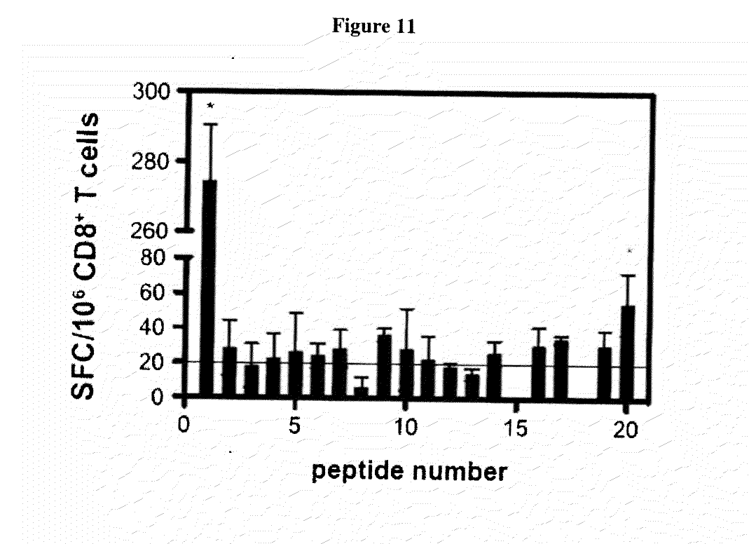

The proteome of the DENV2 strain, S221, was inspected for the presence of peptides predicted to bind HLA A*0201 with high affinity. A total of 68 potential H-2^{b} binding peptides were identified. HLA A*0201 transgenic mice were infected intravenously (i.v.) with 10¹¹ GE of the DENV2 strain, S221. Seven days post-infection, splenocytes were harvested and CD8⁺ T cells isolated. CD8⁺ T cells (1.75×10⁵) were stimulated with HLA-A*0201-restricted Jurkat cells and 1 μg/ml of individual S221-derived A*02-predicted binding peptides, and IFN-γ ELISPOT was performed. Referring to FIG. 11, the data are expressed as the mean number of net spot-forming cells (SFC) per 10⁶ CD8⁺ T cells. The top 20 predicted epitopes, which include the two positive peptides identified, RLITVNPIV (SEQ ID NO:13) and KLAEAIFKL (SEQ ID NO:14), are shown in FIG. 11 (indicated with an asterisk). The criteria for positivity were a stimulation index of ≧2.0, p<0.05 when compared with an irrelevant control peptide, and net SFC/10⁶ cells of ≧20. These results validate that HLA transgenic mice can be used to successfully identify DENV HLA-restricted epitopes.

What is claimed:

1. A peptide comprising or consisting of a subsequence or portion of Dengue virus (DV) structural core (C), membrane (M) or envelope (E) polypeptide sequence, or an amino acid substitution thereof, wherein the subsequence or portion elicits an anti-DV CD8⁺ T cell response.

2. The peptide of claim 1, wherein the Dengue virus (DV) structural core (C), membrane (M) or envelope (E) polypeptide sequence is identical to or derived from a DENV1, DENV2, DENV3 or DENV4 serotype.

3. The peptide of claim 1, wherein the structural subsequence or portion comprises a subsequence or portion of: Core, MNNQRKKARNTPENMLKRERNRVSTVQQLTKRFSLGMLQGRGPLKLFMALVAFLR ELTIPPTAGILKRWGTIKKSKAINVLRGERKEIGRMLNILNRRRRTAGMIIMLIPTVMA; Membrane, FHLTTRNGEPHMIVSRQEKGKSLLEKTGDGVNMCTLMAMDLGELCEDTITYKCPLL RQNEPEDIDCWCNSTSTWVTYGTCTTTGEHRREKRSVALVPHVGMGLETRTETWM SSEGAWKHAQRIETWILRHPGFTIMAAILAYTIGTTHFQRALIFILLTAVAPSMT; Envelope, MRCIGISNRDFVEGVSGGSWVDIVLEHGSCVTTMAKNKPTLDEELIKTEAKQSATLR KYCIEAKLTNTTTESRCPTQGEPSLNEEQDKRFVCKHSMVDRGWGNGCGLFGKGGI VTCAMFTCKKNMKGKVVQPENLEYTIVITPFISGEEHAVGNDTGKHGKEIKITPQSSI TEAELTGYGTVTMECSPRTGLDFNEMVLLQMENKAWLVHRQWFLDLPLPWLPGA DTQGSNWIQKETLVTFKNPHAKKQDVVVLGSQEGAMHTALTGATEIQMSSGNLLF TGHLKCRLRMDKLQLKGMSYSMCTGKEKVVKEIAETQHGTIVIRVQYEGDGSPCKI PFEIMDLEKRHVLGRLITVNPIVTEKDSPVNIEAEPPFGDSYIIIGVEPGQLKLNWFKK GSSIGQMLETTMRGAKRMAILGDTAWDFGSLGGVFTSIGKALHQVFGAIYGAAFSG VSWTMKILIGVIITWIGMNSRSTSLSVSLVLVGVVTLYLGVMVQA; or an Envelope sequence with a substitution of E₁₂₄ N→D; or E₁₂₈ K→E.

4. The peptide of claim 1, wherein the structural protein subsequence or portion comprises or consists of a sequence set forth as: GMLQGRGPL (SEQ ID NO:1); VAFLRFLTI (SEQ ID NO:2); RALIFILL (SEQ ID NO:3); MTMRCIGI (SEQ ID NO:4); VSWTMKIL (SEQ ID NO:5); or RLITVNPIV (SEQ ID NO: l3), or a subsequence thereof or an amino acid substitution thereof.

5. A peptide comprising or consisting of a subsequence or portion of Dengue virus (DV) non-structural (NS) NS2A, NS4B or NS5 polypeptide sequence, or an amino acid substitution thereof, wherein the subsequence or portion elicits an anti-DV CD8⁺ T cell response.

6. The peptide of claim 5, wherein the Dengue virus (DV) non-structural (NS) NS2A, NS4B or NS5 polypeptide sequence is identical to or derived from a DENV1, DENV2, DENV3 or DENV4 serotype.

7. The peptide of claim 5, wherein the non-structural (NS) subsequence or portion comprises a subsequence or portion of: NS1, ADSGCVVSWKNKELKCGSGIFITDNVHTWTEQYKFQPESPSKLASAIQKAHEEGICG IRSVTRLENLMWKQITPELNHILSENEVKLTIMTGDIKGIMQAGKRSLRPQPTELKYS WKTWGKAKMLSTESHNQTFLIDGPETAECPNTNRAWNSLEVEDYGFGVFTTNIWL KLREKQDVFCDSKLMSAAIKDNRAVHADMGYWIESALNDTWKIEKASFIEVKSCH WPKSHTLWSNEVLESEMIIPKNFAGPVSQHNYRPGYHTQTAGPWHLGKLEMDFDFC EGTTVVVTEDCGNRGPSLRTTTASGKLITEWCCRSCTLPPLRYRGEDGCWYGMEIRP LKEKEENLVNSLVTA; NS2A, GHGQIDNFSLGVLGMALFLEEMLRTRVGTKHAILLVAVSFVTLITGNMSFRDLGRV MVMVGATMTDDIGMGVTYLALLAAFKVRPTFAAGLLLRKLTSKELMMTTIGIVLLS QSTIPETILELTDALALGMMVLKMVRKMEKYQLAVTIMAILCVPNAVILQNAWKVS CTILAVVSVSPLFLTSSQQKADWIPLALTIKGLNPTAIFLTTLSRTNKKR; NS2B, SWPLNEAIMAVGMVSILASSLLKNDIPMTGPLVAGGLLTVCYVLTGRSADLELERA ADVKWEDQAEISGSSPILSITISEDGSMSIKNEEEEQTLTILIRTGLLVISGLFPVSLPITA AAWYLWEVKKQR; NS3, AGVLWDVPSPPPVGKAELEDGAYRIKQKGILGYSQIGAGVYKEGTFHTMWHVTRG AVLMHKGKRIEPSWADVKKDLISYGGGWKLEGEWKEGEEVQVLALEPGKNPRAV QTKPGLFKTNAGTIGAVSLDFSPGTSGSPIIDKKGKVVGLYGNGVVTRSGAYVSAIA QTEKSIEDNPEIEDDIFRKRKLTIMDLHPGAGKTKRYLPAIVREAIKRGLRTLILAPTR VVAAEMEEALRGLPIRYQTPAIRAEHTGREIVDLMCHATFTMRLLSPVRVPNYNLII MDEAHFTDPASIAARGYISTRVEMGEAAGIFMTATPPGSRDPFPQSNAPIMDEEREIP ERSWSSGHEWVTDFKGKTVWFVPSIKAGNDIAACLRKNGKKVIQLSRKTFDSEYVK TRTNDWDFVVTTDISEMGANFKAERVIDPRItCMKPVILTDGEERVILAGPMINTHSS AAQRRGRIGRNPKNENDQYIYMGEPLENDEDCAHWKEAKMLLDNINTPEGIIPSMF EPEREKVDAIDGEYRLRGEARKTFVDLMRRGDLPVWLAYRVAAEGINYADRRWCF DGIKNNQILEENVEVEIWTKEGERKKLKPRWLDARIYSDPLALKEFKEFAAGRK; NS4A, SLTLSLITEMGRLPTFMTQKARDALDNLAVLHTAEAGGRAYNHALSELPETLETLLL LTLLATVTGGIFLELMSGRGIGKMTLGMCCIFFASILLWYAQIQPIIWIAASIILEFFLIV LLIPEPEKQRTPQDNQLTYVVIAILTVVAATMA; NS4B, NEMGFLEKTKKDLGLGSITTQQPESNILDIDLRPASAWTLYAVATITVTPMLRHSIEN SSVNVSLTAIANQATVLMGLGKGWPLSKMDIGVPLLAIGCYSQVNPITLTAALFLLV AHYAIIGPGLQAKATREAQKRAAAGIMKNPTVDGITVIDLDPIPYDPKFEKQLGQVM LLVLCVTQVLMMRITWALCEALTLATGPISTLWEGNPGREWNTTIAVSMANIFRGS YLAGAGLLFSIMKNTTNTRR; or NS5, GTGNIGETLGEKWKSRLNALGKSEFQIYKKSGIQEVDRTLAKEGIKRGETDHHAVSR GSAKLRWFVERNMVTPEGKVVDLGCGRGGWSYYCGGLKNVREVKGLTKGGPGHE EPIPMSTYGWNLVRLQSGVDVFFTPPEKCDTLLCDIGESSPNPTVEAGRTLRVLNLVE NWLNNNTQFCIKVLNPYMPSVIEKMEALQRKYGGALVRNPLSRNSTIIEMYWVSNA SGNIVSSVNMISRMLINRFTMRHKKATYEPDVDLGSGTRNIGIESEIPNLDIIGKRIEKI KQEHETSWHYDQDHPYKTWAYHGSYETKQTGSASSMVNGVVRLLTKPWDVVPM VTQMAMTDTTPFGQQRVFKEKVDTRTQEPKEGTKKLMKITAEWLWKELGKKKTP RMCTREEFTRKVRSNAALGAIFTDENKWKSAREAVEDSRFWELVDKERNLHLEGK CETCVYNMMGKREKKLGEFGKAKGSRAIWYMWLGARFLEFEALGFLNEDHWFSR ENSLSGVEGEGLHKLGYILRDVSKKEGGAMYADDTAGWDTRITLEDLKNEEMVTN HMEGEHKKLAEAIFKLTYQNKVVRVQRPTPRGTVMDIISRRDQRGSGQVGTYGLNT FTNMEAQLIRQMEGEGVFKSIQHLTVTEEIAVQNWLARVGRERLSRMAISGDDCVV KPLDDRFASALTALNDMGKVRKDIQQWEPSRGWNDWTQVPFCSHHFHELIMKDGR VLVVPCRNQDELIGRARISQGAGWSLRETACLGKSYAQMWSLMYFHRRDLRLAAN AICSAVPSHWVPTSRTTWSIHAKHEWMTAEDMLTVWNRVWIQENPWMEDKTPVE SWEEIPYLGKREDQWCGSLIGLTSRATWAKNIQTAINQVRSLIGNEEYTDYMPSMKR FRREEEEAGVLW.

8. The peptide of claim 5, wherein the non-structural (NS) protein comprises or consists of a sequence set forth as: FSLGVLGM (SEQ ID NO:6); VAVSFVTLI (SEQ ID NO:7); LAVTIMAIL (SEQ ID NO:8); TAIANQATV (SEQ ID NO:9); TAIANQATV (SEQ ID NO:10); YSQVNPITL (SEQ ID NO:11); RMLINRFTM (SEQ ID NO:12); or KLAEAIFKL (SEQ ID NO:14), a subsequence thereof or an amino acid substitution thereof.

9. The peptide of claim 4 or 8, wherein said amino acid substitution is 1-2, 2-3, 3-4 or 5-6 a conservative, non-conservative, or conservative and non-conservative amino acid substitutions.

10. The peptide of claim 1 or 5, wherein the polypeptide is isolated or purified.

11. The peptide of claim 1 or 5, wherein said an anti-DV CD8⁺ T cell response comprises increased IFN-gamma or TNF-alpha production by CD8⁺ T cells.

12. The peptide of claim 1 or 5, wherein said subsequence or portion of Dengue virus (DV) structural or non-structural (NS) sequence is from about 5 to 300 amino acids in length, provided that said subsequence or portion is at least one amino acid less in length than the full-length structural sequence or the non-structural (NS) sequence.

13. The peptide of claim 1 or 5, wherein said subsequence or portion of Dengue virus (DV) structural or non-structural (NS) sequence is from about 5 to 15, 20 to 25, 25, to 50, 50 to 100, 100 to 150, 150 to 200, or 200 to 300 amino acids in length, provided that said subsequence or portion is at least one amino acid less in length than the full-length structural sequence or the non-structural (NS) sequence.

14. A composition comprising the peptide of claim 1 or 5.

15. A pharmaceutical composition comprising the peptide of claim 1 or 5.

16. The composition of claim 14 or 15, wherein the composition is a liquid or solid.

17. The composition of claim 14 or 15, further comprising an adjuvant.

18. A nucleic acid encoding the peptide of claim 1 or 5.

19. A host cell that expresses the peptide of claim 1 or 5.

20. A method of providing a subject with protection against a Dengue virus (DV) infection or pathology, or one or more physiological conditions, disorders, illness, diseases or symptoms caused by or associated with virus infection or pathology, comprising administering to a subject an amount of a Dengue virus (DV) T cell epitope sufficient to provide the subject with protection against the Dengue virus (DV) infection or pathology, or one or more physiological conditions, disorders, illness, diseases or symptoms caused by or associated with the virus infection or pathology.

21. A method of treating a subject for a Dengue virus (DV) infection, comprising administering to a subject an amount of a Dengue virus (DV) T cell epitope sufficient to treat the subject for the Dengue virus (DV) infection.

22. The method of claim 20, wherein the Dengue virus comprises a DENV1, DENV2, DENV3 or DENV4 serotype.

23. The method of claim 20, wherein the Dengue virus T cell epitope is a structural or non-structural (NS) protein.

24. The method of claim 20, wherein the Dengue virus T cell epitope comprises or consists of a subsequence or portion of Dengue virus C, M or E proteins.

25. The method of claim 23, wherein the structural protein comprises or consists of a peptide sequence set forth as: GMLQGRGPL (SEQ ID NO:1); VAFLRFLTI (SEQ ID NO:2); RALIFILL (SEQ ID NO:3); MTMRCIGI (SEQ ID NO:4); VSWTMKIL (SEQ ID NO:5); or RLITVNPIV (SEQ ID NO:13), a subsequence thereof or an amino acid substitution thereof.

26. The method of claim 20, wherein the Dengue virus T cell epitope comprises or consists of a subsequence or portion of NS2A, NS4B or NS5 proteins.

27. The method of claim 23, wherein the non-structural (NS) protein comprises or consists of a peptide sequence set forth as: FSLGVLGM (SEQ ID NO:6); VAVSFVTLI (SEQ ID NO:7); LAVTIMAIL (SEQ ID NO:8); TAIANQATV (SEQ ID NO:9); TAIANQATV (SEQ ID NO:10); YSQVNPITL (SEQ ID NO:1 1); RMLINRFTM (SEQ ID NO:12); or KLAEAIFKL (SEQ ID NO:14), a subsequence thereof or an amino acid substitution thereof.

28. The method of claim 20, wherein the Dengue virus (DV) infection is acute.

29. The method of claim 20, wherein the subject is a mammal.

30. The method of claim 20, wherein the subject is a human.

31. The method of claim 20, wherein the treatment reduces Dengue virus (DV) titer, increases or stimulates Dengue virus (DV) clearance, reduces or inhibits Dengue virus (DV) proliferation, reduces or inhibits increases in Dengue virus (DV) titer or Dengue virus (DV) proliferation, reduces the amount of a Dengue virus (DV) protein or the amount of a Dengue virus (DV) nucleic acid, or reduces or inhibits synthesis of a Dengue virus (DV) protein or a Dengue virus (DV) nucleic acid.

32. The method of claim 20, wherein the treatment reduces one or more physiological conditions, disorders, illness, diseases or symptoms caused by or associated with Dengue virus (DV) infection or pathology.

33. The method of claim 20, wherein the treatment improves one or more physiological conditions, disorders, illness, diseases or symptoms caused by or associated with Dengue virus (DV) infection or pathology.

34. The method of claim 32, wherein the symptom is a fever, rash, headache, pain behind the eyes, muscle or joint pain, nausea, vomiting, or loss of appetite.

35. The method of claim 20, wherein the treatment reduces or ameliorates an adverse complication associated with Dengue virus (DV) infection or pathology.

36. The method of claim 20, wherein the Dengue virus (DV) T cell epitope is administered prior to, substantially contemporaneously with or following exposure to or infection of the subject with Dengue virus (DV).

37. The method of claim 20, wherein the a plurality of Dengue virus (DV) T cell epitopes are administered prior to, substantially contemporaneously with or following exposure to or infection of the subject with Dengue virus (DV).

38. The method of claim 20, wherein the Dengue virus (DV) T cell epitope is administered within 2-72 hours, 2-48 hours, 4-24 hours, 4-18 hours, or 6-12 hours after a rash develops.

39. A method of inducing, increasing, promoting or stimulating anti-Dengue virus (DV) activity of CD8⁺ T cells in a subject, comprising administering to a subject an amount of a Dengue virus (DV) T cell epitope sufficient to induce, increase, promote or stimulate anti-Dengue virus (DV) activity of CD8⁺ T cells in the subject.

40. The method of claim 39, wherein the CD8⁺ T cells produce IFN gamma, TNF-alpha, IL-1alpha, IL-6 or IL-8.

41. A method of vaccinating a subject against a Dengue virus (DV) infection, comprising administering to a subject an amount of a Dengue virus (DV) T cell epitope sufficient to vaccinate the subject against the Dengue virus (DV) infection.

42. The method of claim 41, wherein the method provides the subject with protection against one or more physiological conditions, disorders, illness, diseases or symptoms caused by or associated with Dengue virus (DV) infection or pathology.

43. The method of claim 41, wherein the Dengue virus T cell epitope is a structural or non-structural (NS) protein.

44. The method of claim 41, wherein the Dengue virus T cell epitope comprises or consists of a subsequence or portion of Dengue virus C, M or E proteins.

45. The method of claim 43, wherein the structural protein comprises or consists of a peptide sequence set forth as: GMLQGRGPL (SEQ ID NO:1); VAFLRFLTI (SEQ ID NO:2); RALIFILL (SEQ ID NO:3); MTMRCIGI (SEQ ID NO:4); VSWTMKIL (SEQ ID NO:5); or RLITVNPIV (SEQ ID NO:13), a subsequence thereof or an amino acid substitution thereof.

46. The method of claim 41, wherein the Dengue virus T cell epitope comprises or consists of a subsequence or portion of NS2A, NS4B or NS5 proteins.

47. The method of claim 43, wherein the non-structural (NS) protein comprises or consists of a peptide sequence set forth as: FSLGVLGM (SEQ ID NO:6); VAVSFVTLI (SEQ ID NO:7); LAVTIMAIL (SEQ ID NO:8); TAIANQATV (SEQ ID NO:9); TAIANQATV (SEQ ID NO:10); YSQVNPITL (SEQ ID NO:11); RMLINRFTM (SEQ ID NO:12); or KLAEAIFKL (SEQ ID NO:14), a subsequence thereof or an amino acid substitution thereof.

48. The method of claim 41, wherein the Dengue virus (DV) infection is acute.

49. The method of claim 41, wherein the subject is a mammal.

50. The method of claim 41, wherein the subject is a human.

51. The method of claim 41, wherein the vaccinating reduces or inhibits susceptibility to Dengue virus (DV) infection or pathology.

52. The method of claim 41, wherein the vaccinating reduces or inhibits one or more physiological conditions, disorders, illness, diseases or symptoms caused by or associated with Dengue virus (DV) infection or pathology.

53. The method of claim 41, wherein the vaccinating improves one or more physiological conditions, disorders, illness, diseases or symptoms caused by or associated with Dengue virus (DV) infection or pathology.

54. The method of claim 41, wherein the vaccinating reduces or ameliorates an adverse complication associated with Dengue virus (DV) infection or pathology.

55. The method of claim 41, wherein the Dengue virus (DV) T cell epitope is administered prior to exposure to or infection of the subject with Dengue virus (DV).

56. The method of claim 41, wherein a plurality of Dengue virus (DV) T cell epitopes are administered prior to, substantially contemporaneously with or following exposure to or infection of the subject with Dengue virus (DV).
